(19) United States
(12) Patent Application Publication
(10) Pub. No.: US 2010/0105764 A1
(43) Pub. Date: Apr. 29, 2010

(54) USE

(76) Inventors: Michael John Reed, Slough (GB); Atul Purohit, Slough (GB); Paul Alexander Foster, Slough (GB); Simon Paul Newman, Slough (GB); Lok Wai Lawrence Woo, Slough (GB); Barry Victor Lloyd Potter, Slough (GB); Gillian Reed, Slough (GB)

Correspondence Address: FROMMER LAWRENCE & HAUG, 745 FIFTH AVENUE- 10TH FL., NEW YORK, NY, 10151, US

(21) Appl. No.: 12/475,910

(22) Filed: Jun. 1, 2009

Related U.S. Application Data
(63) Continuation-in-part of application No. PCT/GB2007/004612, filed on Nov. 30, 2007.

(30) Foreign Application Priority Data
Dec. 1, 2006 (GB) 0624105.3

(51) Int. Cl. A61K 31/366; A61P 35/00

(52) U.S. Cl. 514/455

(57) ABSTRACT

There is provided a method for the manufacture of a medicament for the treatment of cancer comprising a compound capable of inhibiting a steroid sulphatase enzyme (E.C.3.1.6.2), wherein the cancer is of a type, in which the cancer cells overexpress aromatase enzyme.

### INCORPORATION BY REFERENCE

This application is a continuation-in-part of international patent application Serial No. PCT/GB2007/004612 filed Nov. 30, 2007, which published as PCT Publication No. WO 2008/065428 on Jun. 5, 2008, which claims priority from Great Britain Patent Application No. 0624105.3 filed Dec. 1, 2006.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention also relates to the use of a compound or composition containing the same in therapy applications.

### BACKGROUND OF THE INVENTION

Evidence suggests that oestrogens are the major mitogens involved in promoting the growth of tumours in endocrine-dependent tissues, such as the breast and endometrium. Although plasma oestrogen concentrations are similar in women with or without breast cancer, breast tumour oestrone and oestradiol levels are significantly higher than in normal breast tissue or blood. In situ synthesis of oestrogen is thought to make an important contribution to the high levels of oestrogens in tumours and therefore inhibitors, in particular specific inhibitors, of oestrogen biosynthesis are of potential value for the treatment of endocrine-dependent tumours.

Over the past two decades, there has been considerable interest in the development of inhibitors of the aromatase pathway—which converts the androgen precursor androstenedione to oestrone. However, there is now evidence that the oestrone sulphatase (E1-STS) pathway, i.e. the hydrolysis of oestrone sulphate to oestrone (E1S to E1), and aromatase (i.e. conversion of androstenedione to oestrone) account for the production of oestrogens in breast tumours.

FIGS. 1 and 2 are schematic diagrams showing some of the enzymes involved in the in situ synthesis of oestrone from oestrone sulphate, oestradiol and androstenedione.

In FIG. 2, which schematically shows the origin of oestrogenic steroids in postmenopausal women, “ER” denotes Oestrogen Receptor, “DHEA-S” denotes Dehydroepiandrosterone-Sulphate, “Adiol” denotes Androstenediol, “E1-STS” denotes Oestrone Sulphatase, “DHEA-STS” denotes DHEA-sulphatase, “Adiol-STS” denotes Adiol Sulphatase, and “17B-HSD” denotes Oestradiol 17B-hydroxysteroid dehydrogenase.

As can be seen, the main two enzymes that are involved in the peripheral synthesis of oestrogens are the aromatase enzyme and the enzyme oestrone sulphatase.

In short, the aromatase enzyme converts androstenedione, which is secreted in large amounts by the adrenal cortex, to oestrone. Recent reports have suggested that some flavones could inhibit aromatase activity.

Much of the oestrone so formed, however, is converted to oestrone sulphate (E1S) and there is now a considerable body of evidence showing that E1S in plasma and tissue acts as a reservoir for the formation of oestrone by the action of oestrone sulphatase.

In this regard, it is now believed that the oestrone sulphatase (E1-STS) pathway—i.e. the hydrolysis of oestrone sulphate to oestrone (E1S to E1) is a major source of oestrogen in breast tumours. This theory is supported by a modest reduction of plasma oestrogen concentration in postmenopausal women with breast cancer treated by aromatase inhibitors, such as aminoglutethimide and 4-hydroxyandrostenedione and also by the fact that plasma E1S concentration in these aromatase inhibitor-treated patients remains relatively high. The long half-life of E1S in blood (10-12 h) compared with the unconjugated oestrogens (20 min) and high levels of steroid sulphatase activity in liver and, normal and malignant breast tissues, also lend support to this theory.

Thus, oestrogen formation in malignant breast and endometrial tissues via the sulphatase pathway makes a major contribution to the high concentration of oestrogens which are present in these tumours. However, inhibition of both the aromatase and sulphatase pathways could offer considerable therapeutic benefit.

PCT/GB92/01587 teaches novel steroid sulphatase inhibitors and pharmaceutical compositions containing them for use in the treatment of oestrone dependent tumours, especially breast cancer. These steroid sulphatase inhibitors are sulphamate esters, such as N,N-dimethyl oestrone-3-sulphamate and, preferably, oestrone-3-sulphamate (otherwise known as “EMATE”). EMATE has the following structure:

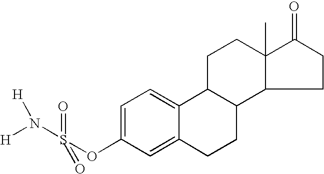

It is known that EMATE is a potent E1-STS inhibitor as it displays more than 99% inhibition of E1-STS activity in intact MCF-7 cells at 0.1 nM. EMATE also inhibits the E1-STS enzyme in a time- and concentration-dependent manner, indicating that it acts as an active site-directed inactivator. Although EMATE was originally designed for the inhibition of E1-STS, it also inhibits dehydroepiandrosterone sulphatase (DHEA-STS), which is an enzyme that is believed to have a pivotal role in regulating the biosynthesis of the oestrogenic steroid androstenediol. Also, there is now evidence to suggest that androstenediol may be of even greater importance as a promoter of breast tumour growth. EMATE is also active in vivo as almost complete inhibition of rat liver E1-STS (99%) and DHEA-STS (99%) activities resulted when it is administered either orally or subcutaneously. In addition, EMATE has been shown to have a memory enhancing effect in rats. Studies in mice have suggested an association between DHEA-STS activity and the regulation of part of the immune response. It is thought that this may also occur in humans. The bridging O-atom of the sulphamate moiety in EMATE is important for inhibitory activity. Thus, when the 3-O-atom is replaced by other heteroatoms as in oestrone-3-N-sulphamate and oestrone-3-S-sulphamate, these analogues are weaker non-time-dependent inactivators.

In addition to oestrone, the other major steroid with oestrogenic properties which is produced by postmenopausal women is androstenediol (see FIG. 2).

Androstenediol, although an androgen, can bind to the oestrogen receptor (ER) and can stimulate the growth of ER positive breast cancer cells and the growth of carcinogen-induced mammary tumours in the rat. Importantly, in postmenopausal women 90% of the androstenediol produced originates from the androgen dehydroepiandrosterone sulphate (DHEA-S) which is secreted in large amounts by the adrenal cortex. DHEA-S is converted to DHEA by DHEA sulphatase, which may be the same as, or different from, the enzyme, oestrone sulphatase, which is responsible for the hydrolysis of E1S.

During the last 10-15 years considerable research has also been carried out to develop potent aromatase inhibitors, some of which are now marketed. However, in three recent reports of postmenopausal women with breast cancer who received aromatase inhibitor therapy, plasma E1S concentrations remained between 400-1000 pg/ml.

In summation therefore in situ synthesis of oestrogen is thought to make an important contribution to the high levels of oestrogens in tumours and therefore specific inhibitors of oestrogen biosynthesis are of potential value for the treatment of endocrine-dependent tumours.

Moreover, even though oestrogen formation in malignant breast and endometrial tissues via the sulphatase pathway makes a major contribution to the high concentration of oestrogens, there are still other enzymatic pathways that contribute to in vivo synthesis of oestrogen.

The present invention seeks to provide novel compounds suitable for the inhibition of steroid sulphatase activity and aromatase activity.

Citation or identification of any document in this application is not an admission that such document is available as prior art to the present invention.

### SUMMARY OF THE INVENTION

In a first aspect the present invention provides use of a compound capable of inhibiting a steroid sulphatase enzyme (E.C.3.1.6.2) in the manufacture of a medicament for the treatment of cancer, wherein the cancer is of a type in which the cancer cells overexpress aromatase enzyme.

In a second aspect the present invention provides use of a compound capable of inhibiting a steroid sulphatase enzyme (E.C.3.1.6.2) in the manufacture of a medicament for the treatment of a tumour, wherein the tumour is formed from cancer cells overexpress aromatase enzyme.

In a third aspect the present invention provides use of a compound capable of inhibiting a steroid sulphatase enzyme (E.C.3.1.6.2) in the manufacture of a medicament for the treatment of a proliferative disease, wherein the proliferative disease is of a type in which the proliferative cells overexpress aromatase enzyme.

In a fourth aspect the present invention provides use of a compound capable of inhibiting a steroid sulphatase enzyme (E.C.3.1.6.2) in the manufacture of a medicament for the treatment of cancer associated with overexpressed aromatase enzyme.

In a fifth aspect the present invention provides use of a compound capable of inhibiting a steroid sulphatase enzyme (E.C.3.1.6.2) in the manufacture of a medicament for the treatment of a tumour, wherein tumour growth is associated with overexpressed aromatase enzyme.

The present invention is based on the surprising finding that steroid sulphatase inhibitors may inhibit the growth of cancers, tumours and proliferative diseases in which the aromatase enzyme is overexpressed. In these conditions, the overexpression of aromatase enzyme results in the rapid in vivo synthesis of hormones which may stimulate the development of the disease. For example in hormone dependent tumours, androstenediol (Adiol) and estradiol (E2) may stimulate the growth of the tumour. As can be seen from FIG. 1, the in vivo pathway for the synthesis of E2 is via E1 whereas Adiol originates from DHEAS. Oestrone is converted in this pathway to its inactive precursor estrone sulfate, and in systems overexpressing aromatase enzyme, this conversion is rapid. We have found that providing an inhibitor of steroidal sulphatase, the conversion back from estrone sulfate to estrone is blocked and the reactivation of the stimulatory hormones is prevented. Because of the overexpression of aromatase enzyme the effect is particularly pronounced when compared to cells having a normal level of aromatase enzyme expression.

It will be understood by one skilled in the art that by the term “overexpress aromatase enzyme” (and its derivative terms such as “aromatase enzyme overexpression”, “overexpressed aromatase enzyme”) it is meant that the expression is higher than that of the wild type of the cell in question.

For ease of reference, these and further aspects of the present invention are now discussed under appropriate section headings. However, the teachings under each section are not necessarily limited to each particular section.

It is noted that in this disclosure and particularly in the claims and/or paragraphs, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

These and other embodiments are disclosed or are obvious from and encompassed by, the following Detailed Description.

### BRIEF DESCRIPTION OF THE DRAWINGS

The following detailed description, given by way of example, but not intended to limit the invention solely to specific embodiments described, may be understood in conjunction with the accompanying Figures, incorporated herein by reference, in which:

FIG. 1 is a schematic diagram showing some of the enzymes involved in the in situ synthesis of oestrone from oestrone sulphate, oestradiol and androstenedione.

FIG. 2 schematically shows the origin of oestrogenic steroids in postmenopausal women, where “ER” denotes Oestrogen Receptor, “DHEA-S” denotes Dehydroepiandrosterone-Sulphate, “Adiol” denotes Androstenediol, “E1-STS” denotes Oestrone Sulphatase, “DHEA-STS” denotes DHEA-sulphatase, “Adiol-STS” denotes Adiol Sulphatase, and “17B-HSD” denotes Oestradiol 17B-hydroxysteroid dehydrogenase.

FIG. 3 shows that steroid sulfatase (STS) is responsible for the hydrolysis of estrone sulfate (E1S) and dehydroepiandrosterone sulfate (DHEAS) to estrone (E1) and DHEA respectively, which can be reduced in the body to estradiol (E2) and androstenediol (Adiol), both of which have potent estrogenic properties (reviewed in Reed et al., Endocrine Reviews, 256:171-202, 2005).

FIG. 4 shows that androgen stimulated growth is blocked by an anti-oestrogen and that an aromatase inhibitor failed to block DHEAS stimulated growth whereas a steroid sulphatase inhibitor did.

FIG. 5a shows data in respect of MCF-7 cells transfected with aromatase and the growth of which is stimulated with androstenedione (A4).

FIG. 5b shows data in respect of MCF-7 cells transfected with steroid sulphatase (STS) and the growth of which is stimulated with E1S.

FIG. 6 shows that oral administration of letrozole (0.1 mg/kg) resulted in some reduction of tumour growth, the second generation STS inhibitor, STX213, significantly reduced the growth of MCF-7_{WT} and MCF-7_{STS}+MCF-7_{AROM} tumours.

FIG. 6a shows that STX213 (a 2nd generation STS inhibitor) inhibited growth of MCF-7 wt and tumors derived from MCF-7AROM and MCF-7STS to a greater extent than Letrozole.

FIG. 6b provides data for Letrozole 0.1 mg/kg p.o. and STX213 10 mg/kg p.o. ( 5/7 per week).

FIG. 7 shows that, at the dose tested, STX213 was devoid of any toxicity as shown by its lack of effect on body weight.

FIG. 8 shows that tumours derived from MCF-7_{STS} or MCF-7_{AROM} cells grew in the presence of A4 plus E2S but no growth occurred in their absence.

FIG. 9 shows that oral administration of letrozole (0.1 mg/kg) resulted in significant inhibition in the growth of tumours derived from MCF-7_{AROM} cells but did not affect the growth of tumours derived from MCF-7_{STS} cells.

FIG. 10 shows that oral administration of the STS inhibitor STX64 resulted in significant inhibition of tumour growth derived from not only from MCF 7_{STS} cells, as expected, but also MCF-7_{AROM} cells.

FIG. 11 shows that dosing with the combination of letrozole plus STX64 did not improve the tumour growth inhibition achieved with STX64 alone.

FIG. 12 shows that STX64 or letrozole, alone or in combination, were well tolerated with no effects on animal weight being detected.

### DETAILED DESCRIPTION

As discussed herein the use of a compound capable of inhibiting a steroid sulphatase enzyme (E.C.3.1.6.2) in the manufacture of a medicament for the treatment of cancer, wherein the cancer is of a type in which the cancer cells overexpress aromatase enzyme.

In preferred aspects the cancer is selected from breast cancer, ovarian cancer, prostate cancer, and endometrial cancer. Preferably the cancer is breast cancer.

As will be understood by one skilled in the art the nature of the blocking of stimulatory hormones is best utilised in the treatment of hormone dependent conditions such as hormone dependent cancer. The conditions/cancer may be dependent on one hormone or may be dependent on multiple hormones. In one preferred aspect the condition/cancer is oestrogen dependent.

In a highly preferred aspect the cancer is hormone dependent breast cancer.

In another highly preferred aspect the cancer is oestrogen dependent breast cancer, such as estradiol dependent breast cancer.

### Compound

The compound may be any suitable compound. Classes of suitable compounds will now be described.

### Sulphamate Compounds

Preferably the compound comprises a sulphamate group. In this aspect the compound is referred to as a sulphamate compound.

The term “sulphamate” includes an ester of sulphamic acid, or an ester of an N-substituted derivative of sulphamic acid, or a salt thereof.

The sulphamate group preferably has the formula:

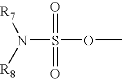

wherein R₇ and R₈ are independently selected from H or a hydrocarbyl group.

Preferably R₇ and R₈ are independently selected from H, alkyl, cycloalkyl, alkenyl, acyl and aryl, or combinations thereof, or together represent alkylene, wherein the or each alkyl or cycloalkyl or alkenyl or aryl optionally contains one or more hetero atoms or groups.

When substituted, the N-substituted compounds of this invention may contain one or two N-alkyl, N-alkenyl, N-cycloalkyl, N-acyl, or N-aryl substituents, preferably containing or each containing a maximum of 10 carbon atoms. When R₇ and/or R₈ is alkyl, the preferred values are those where R₇ and R₈ are each independently selected from lower alkyl groups containing from 1 to 5 carbon atoms, that is to say methyl, ethyl, propyl etc. preferrably both are methyl. When R₇ and/or R₈ is aryl, typical values are phenyl and tolyl (-PhCH₃; o-, m- or p-). Where R₇ and/or R₈ represent cycloalkyl, typical values are cyclopropyl, cyclopentyl, cyclohexyl etc. When joined together R₇ and R₈ typically represent an alkylene group providing a chain of 4 to 6 carbon atoms, optionally interrupted by one or more hetero atoms or groups, e.g.—O— or —NH— to provide a 5-, 6- or 7-membered heterocycle, including, but not limited to, morpholine, pyrrolidine or piperidine.

Within the values alkyl, cycloalkyl, alkenyl, acyl and aryl we include substituted groups containing as substituents therein one or more groups which do not interfere with the sulphatase inhibitory activity of the compound in question. Exemplary non-interfering substituents include, but are not limited to, hydroxy, amino, halo, alkoxy, alkyl and aryl. A non-limiting example of a hydrocarbyl group is an acyl group.

In some embodiments, the sulphamate group may form a ring structure by being fused to (or associated with) one or more atoms in or on the steroidal ring system.

In some embodiments, there may be more than one sulphamate group. By way of example, there may be two sulphamates (i.e. bis-sulphamate compounds).

In some preferred embodiments, at least one of R₇ and R₉ is H.

In some preferred embodiments, each of R₇ and R₉ is H.

In some preferred embodiments if the sulphamate group on the sulphamate compound were to be replaced with a sulphate group to form a sulphate compound then the sulphate compound would be hydrolyzable by a steroid sulphatase enzyme (E.C.3.1.6.2).

In some preferred embodiments if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a Kₘ value of less than 50 mM.

In some preferred embodiments if the sulphamate group on the sulphamate compound were to be replaced with a sulphate group to form a sulphate compound and incubated with a steroid sulphatase enzyme (E.C.3.1.6.2) at a pH 7.4 and 37° C. it would provide a Kₘ value of less than 50 μM.

### Coumarin Based Compounds

In one preferred aspect the compound is a compound in accordance with the teachings of WO 97/30041.

Preferably the compound is of Formula (A),

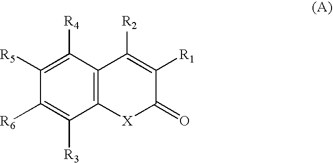

wherein R₁-R₆ are independently selected from H, halo, hydroxy, sulphamate, alkyl and substituted variants or salts thereof; but wherein at least one of R₁-R₆ is a sulphamate group and wherein X is selected from O, NR₉, and CR₁₀R₁₁, wherein R₉ is selected from H and hydrocarbyl, and wherein R₁₀ and R₁₁ are independently selected from H, halo, hydroxy and hydrocarbyl.

Preferably two or more of R₁-R₆ are linked together to form an additional cyclic structure.

Preferably X is O.

Preferably R₁-R₆ are independently selected from H, alkyl and haloalkyl.

Preferably R₁-R₆ are independently selected from H, C₁₋₆ alkyl and C₁₋₆ haloalkyl.

Preferably R₁-R₆ are independently selected from H, C₁₋₃ alkyl and C₁₋₃ haloalkyl.

Preferably R₁-R₆ are independently selected from H, methyl and halomethyl.

Preferably the compound is of Formula (C),

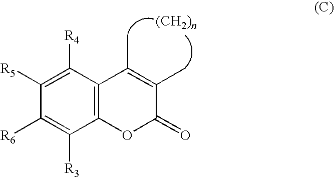

wherein R₃-R₆ are independently selected from H, halo, hydroxy, sulphamate, alkyl and substituted variants or salts thereof; but wherein at least one of R₃-R₆ is a sulphamate group, and wherein n is from 3 to 14. Preferably n is from 3 to 10. More preferably n is 5.

In one preferred aspect R₆ is a sulphamate group.

Particularly preferred compounds are those of the Formulae,

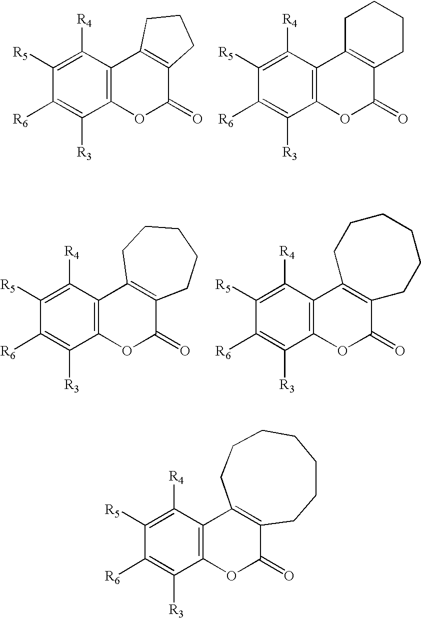

wherein R₃-R₆ are independently selected from H, halo, hydroxy, sulphamate, alkyl and substituted variants or salts thereof; but wherein at least one of R₃-R₆ is a sulphamate group.

Preferably the sulphamate group is as discussed herein and preferably has the formula:

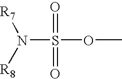

wherein R₇ and R₈ are independently selected from H, alkyl, cycloalkyl, alkenyl, acyl and aryl, or combinations thereof, or together represent alkylene, wherein the or each alkyl or cycloalkyl or alkenyl or optionally contain one or more hetero atoms or groups. More preferably at least one of R₇ and R₈ is H. Yet more preferably each of R₇ and R₈ is H.

In highly preferred aspects the compound is selected from compounds of the Formulae

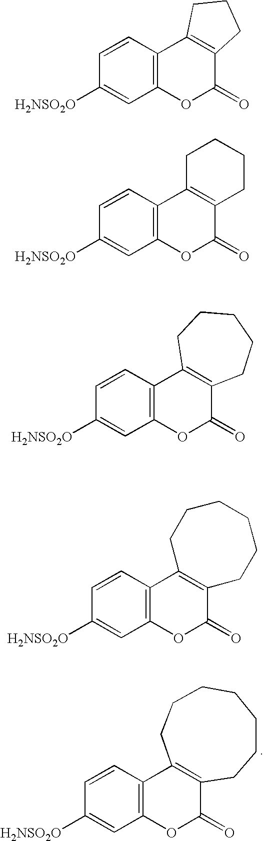

In a very highly preferred aspect the compound is

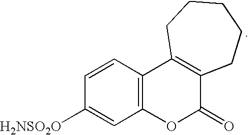

### Arylsulfonamides

In one preferred aspect the compound is a compound in accordance with the teachings of Lehr et al “N-Acyl arylsulfonamides STS inhibitors” 2005 BMCL.

### Cyclic Sulphamates

In one preferred aspect the compound is a compound in accordance with the teachings of one of WO93/05064, U.S. Pat. No. 5,616,574, U.S. Pat. No. 5,830,886, U.S. Pat. No. 6,011,024, U.S. Pat. No. 6,159,960, U.S. Pat. No. 6,187,766, U.S. Pat. No. 6,476,011, U.S. Pat. No. 6,677,325, and U.S. Pat. No. 6,642,397. A typical compound is a compound comprising a steroidal ring structure and a sulphamate group of the formula

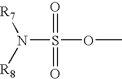

wherein each of R₇ and R₈ is independently selected from H, alkyl, alkenyl, cycloalkyl and aryl; wherein preferably at least one of R₇ and R₈ is H; wherein the compound is an inhibitor of an enzyme having steroid sulphatase activity (E.C.3.1.6.2); and wherein if the sulphamate group on the compound were to be replaced with a sulphate group to form a sulphate compound and incubated with a steroid sulphatase enzyme (E.C.3.1.6.2) at a pH 7.4 and 37° C. it would provide a Kₘ value of less than 50 μM.

### Thiophosphonates

In one preferred aspect the compound is a compound in accordance with the teachings of one of WO91/13083, U.S. Pat. No. 5,281,587, and U.S. Pat. No. 5,344,827. A typical compound is a steroid-3-thiophosphonate of the formula

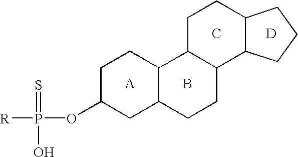

where R is an alkyl group, and the ring system ABCD represents a substituted or unsubstituted saturated or unsaturated steroid nucleus.

### Sulphonates/Phosphonates

In one preferred aspect the compound is a compound in accordance with the teachings of one of WO 93/05063, U.S. Pat. No. 5,604,215, U.S. Pat. No. 5,861,390, and U.S. Pat. No. 6,017,904. A typical compound is a sulphonate or phosphonate compound of the Formula:

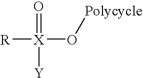

where R is selected from H, alkyl, cycloalkyl, alkenyl and aryl; X is P or S; Y is OH when X is P, and O when X is S; and —O-polycycle represents the residue of a polycyclic alcohol being a polycyclic alcohol the sulphate of which is hydrolysable by enzymes having steroid sulphatase (E.C. 3.1.6.2) activity.

### Steroid Derivatives

In one preferred aspect the compound is a compound in accordance with the teachings of one of WO98/24802 and U.S. Pat. No. 6,642,220. A typical compound is
a sulphamate compound having the Formula;

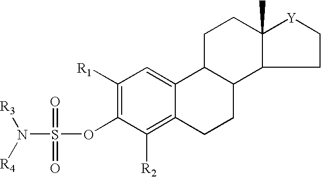

wherein R₁ and/or R₂ is a substituent other than H; wherein R₁ and R₂ may be the same or different but not both being H; each of R₃ and R₄ is independently selected from H, alkyl, cycloalkyl, alkenyl and aryl, wherein at least one of R₃ and R₄ is H; and Y is a suitable linking group (preferably —CH₂— or —C(O)—); OR
a sulphamate compound having the Formula;

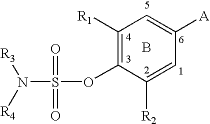

wherein R₁ and optionally R₂ is a substituent other than H; wherein R₁ and R₂ may be the same or different; each of R₃ and R₄ is independently selected from H, alkyl, cycloalkyl, alkenyl and aryl, wherein at least one of R₃ and R₄ is H; and group A is additionally attached to the carbon atom at position 1 of the ring B; OR
a sulphamate compound having the Formula

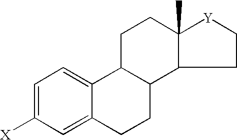

wherein X is a sulphamate group, and Y is CH₂ and optionally any other H attached directly to the ring system is substituted by another group.

### Oximes

In one preferred aspect the compound is a compound in accordance with the teachings of one of WO 99/27936 and U.S. Pat. No. 6,670,353. A typical compound is a sulphamate compound wherein the compound is a polycyclic compound comprising at least two ring components, wherein the polycyclic compound comprises at least one sulphamate group attached to at least one of the ring components, and wherein at least one oxime group is attached to or is part of at least one of the ring components. Such compounds include a sulphamate compound of the formula

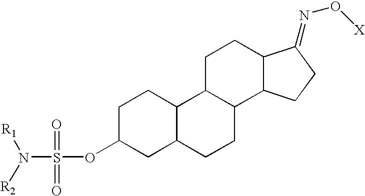

wherein each of R₁ and R₂ is independently selected from H or a hydrocarbyl group, wherein X is H or a hydrocarbyl group.

### Lactones

In one preferred aspect the compound is a compound in accordance with the teachings of WO98/11124. A typical compound is a sulphamate compound wherein the compound is a polycyclic compound comprising at least two ring components, wherein the polycyclic compound comprises at least one sulphamate group attached to at least one of the ring components, and wherein at least one of the ring components of the polycyclic structure is a heterocyclic ring. Such compounds include a sulphamate compound of the formula:

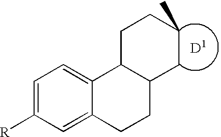

wherein R is a sulphamate group and D¹ represents a heterocyclic ring and/or a six membered ring.

### Halogenated Derivates

In one preferred aspect the compound is a compound in accordance with the teachings of WO01/44268. A typical compound is a compound of the formula

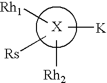

wherein: X is a ring having at least 4 atoms in the ring; K is a hydrocarbyl group; Rh1 is an optional halo group; Rh2 is an optional halo group; at least one of Rh1 and Rh2 is present; Rs is any one of a sulphamate group, a phosphonate group, a thiophosphonate group, a sulphonate group or a sulphonamide group. Such compounds include a compound of the formula

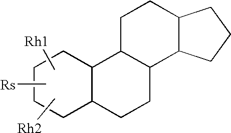

wherein Rh1 is an optional halo group; Rh2 is an optional halo group; at least one of Rh1 and Rh2 is present; Rs is a sulphamate group.

### Sulphanyl Derivatives

In one preferred aspect the compound is a compound in accordance with the teachings of WO02/16394. A typical compound is a compound of the formula

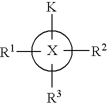

wherein: X is a ring having at least 4 atoms in the ring; K is a hydrocarbyl group; R¹ is an optional group of the formula -L¹-S—R^{1′}, wherein L¹ is an optional linker group and R^{1′} is a hydrocarbyl group; R² is an optional group of the formula -L²-S—R^{2′}, wherein L² is an optional linker group and R^{2′} is a hydrocarbyl group; R³ is any one of a sulphamate group, a phosphonate group, a thiophosphonate group, a sulphonate group or a sulphonamide group; wherein at least one of R¹ and R² is present; and wherein said compound is capable of inhibiting steroid sulphatase (STS) activity and/or is capable of acting as a modulator of cell cycling and/or as a modulator of apoptosis and/or as a modulator of cell growth. Such compounds include a compound of the formula

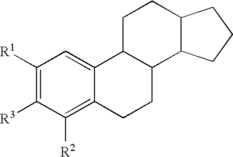

wherein: R¹ is an optional group of the formula -L¹-S—R^{1′}; wherein L¹ is an optional C₁₋₁₀ hydrocarbyl group; R^{1′} is a C₁₋₁₀ hydrocarbyl group; R² is an optional group of the formula -L²-S—R^{2′}; wherein L² is an optional C₁₋₁₀ hydrocarbyl group; R^{2′} is a C₁₋₁₀ hydrocarbyl group; wherein at least one of R¹ and R² is present; R³ is a sulphamate group of the formula (R⁴)(R⁵)N—S(O)(O)—O—; wherein R⁴ and R⁵ are each independently selected from hydrogen, alkyl, cycloalkyl, alkenyl and aryl, or combinations thereof, or together represent alkylene, wherein the or each alkyl or cycloalkyl or alkenyl contain one or more heteroatoms or groups; wherein position 17 of the D ring is optionally substituted by ═O, hydroxy, ethinyl, a hydrocarbyl group, or
i) a sulphamate group of the formula (R⁹)(R¹⁰)N—S(O)(O)—O—
ii) a phosphonate group of the formula (R¹¹)—P(O)(OH)—O—
iii) a thiophosphonate group of the formula (R¹²)—P(S)(OH)—O—
iv) a sulphonate group of the formula (R¹³)—S(O)(O)—O—;
wherein R⁹ and R¹⁰ are each independently selected from hydrogen, alkyl, cycloalkyl, alkenyl and aryl, or combinations thereof, or together represent alkylene, wherein the or each alkyl or cycloalkyl or alkenyl contain one or more heteroatoms or groups; wherein R¹¹, R¹² and R¹³ is hydrogen, alkyl, cycloalkyl, alkenyl and aryl, or combinations thereof, wherein the or each alkyl or cycloalkyl or alkenyl contain one or more heteroatoms or groups; wherein the ring system is optionally substituted by one or more substituents selected from hydroxy, alkyl, alkoxy, alkynyl, and halogen.

### Aryl Substitutions

In one preferred aspect the compound is a compound in accordance with the teachings of WO 02/16393. A typical compound is a compound comprising a steroidal ring system and a group R¹ selected from any one of a sulphamate group, a phosphonate group, a thiophosphonate group, a sulphonate group or a sulphonamide group; wherein the D ring of the steroidal ring system is substituted by a group R² of the formula -L-R³, wherein L is an optional linker group and R³ is an aromatic hydrocarbyl group. Such compounds include a compound of the formula

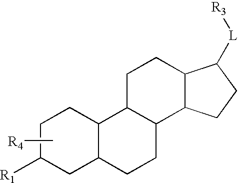

wherein: R¹ is selected from: i) a sulphamate group of the formula (R⁵)(R⁶)N—S(O)(O)—O—; ii) a phosphonate group of the formula (R⁷)—P(O)(OH)—O—, iii) a thiophosphonate group of the formula (R⁸)—P(S)(OH)—O—, iv) a sulphonate group of the formula (R⁹)—S(O)(O)—O—; wherein R⁵ and R⁶ are each independently selected from hydrogen, alkyl, cycloalkyl, alkenyl and aryl, or combinations thereof, or together represent alkylene, wherein the or each alkyl or cycloalkyl or alkenyl contain one or more heteroatoms or groups; wherein R⁷, R⁸ and R⁹ is hydrogen, alkyl, cycloalkyl, alkenyl and aryl, or combinations thereof, wherein the or each alkyl or cycloalkyl or alkenyl contain one or more heteroatoms or groups; L is optionally present and is a C₁₋₁₀ alkyl group; R₃ is a six-membered aromatic ring containing carbon and optionally nitrogen, optionally substituted with a group selected from C₁₋₁₀ alkyl and halogen; R₄ is selected from C₁₋₁₀ alkoxy, C₁₋₁₀ alkyl, or a group of the formula -L⁴-S—R^{4′} wherein L⁴ is optionally present and is a C₁₋₁₀ alkyl; R^{4′} is C₁₋₁₀ alkyl, wherein the ring system is optionally substituted by one or more substituents selected from hydroxy, alkyl, alkoxy, alkynyl, and halogen.

### Multiple Sulphamate Substitution

In one preferred aspect the compound is a compound in accordance with the teachings of WO 02/16392. A typical compound is a compound of the formula

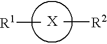

wherein: X is a ring system; R¹ is any one of a sulphamate group, a phosphonate group, a thiophosphonate group, a sulphonate group or a sulphonamide group; R² is any one of a sulphamate group, a phosphonate group, a thiophosphonate group, a sulphonate group or a sulphonamide group; wherein when X is a steroidal structure and both of R¹ and R² are sulphamate groups, the steroidal ring system (X) represents an oestrogen. Such compounds include a compound of the formula

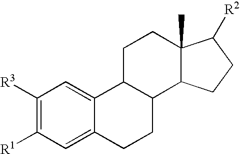

wherein R¹ and R² are sulphamate groups, wherein each sulphamate group is of the formula

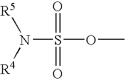

wherein each of R⁴ and R⁵ is independently selected from H and hydrocarbyl; wherein R³ is a hydrocarbyl or oxyhydrocarbyl group; and wherein the ring system may contain one or more hydroxy, alkyl, alkoxy, alkynyl or halogen substituents.

In one preferred aspect the compound is a compound in accordance with the teachings of one of WO 98/42729 and U.S. Pat. No. 6,339,079. A typical compound is a steroid of gonan and D-homogonan type of the formula

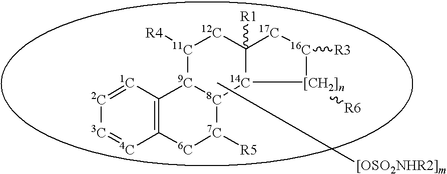

wherein there may be an additional double bond between the C-atoms 9 and 11, 8 and 9, 8 and 14, 14 and 15, 15 and 16, 6 and 7, or 7 and 8, or wherein in each case there are possibly two double bonds between the C-atoms 8, 9, 14, 15 or 8, 9, 7, 6, or which possess a cyclopropane or epoxide group, with α or β orientation, between the C-atoms 14 and 15 or 15 and 16, wherein the C-atoms 2, 3, 4, 6, 7, 11, 12, 15, 16 and/or 17 are unsubstituted or substituted by C₁-C₆-alkyloxy, C₁-C₄-alkyloxy C₁-C₄-alkyloxy, hydroxy-C₁-C₄-alkyloxy, C₁-C₆-alkanoyloxy or tris-(C₁-C₄-alkyl)-silyloxy or hydroxy, wherein, in place of a secondary hydroxy group —CH(OH)— a keto group —C(═O)— can also be present which could be protected in the form of a ketal, thioketal, cyanohydrin, cyanosilyl ether or a geminal hydroxyethinyl group, n=1 or 2, R₁=H, α or β methyl, or α or β ethyl, the sulfamoyloxy residue —OSO₂NHR₂ is located on C-1, -2, -3, -4, -6, -7, -11, -15, -16 and/or -17, as well as on the residues R₄ and/or R₅, R₂=H, C₁-C₅-alkyl, C₁-C₃-alkyl with annelated saturated ring, aryl —C₁-C₃-alkyl, C₁-C₅-alkanoyl, C₃-C₇-cycloalkyl-carbonyl, R₃=H, OH, halogen, pseudohalogen, C₁-C₃-alkyl, C₃-C₇-cycloalkyl, 1′,1′-cycloalkyl or aryl-C₁-C₃-alkyl, R₄=H, aryl or C₁-C₁₂-alkyl, R₅=H, C₁-H₁₂-alkyl or C₁-C₁₂-alkylaryl, R₆=H or halogen, and m=1 to 5, with the stipulation that R₃ is different from H and OH if m is 1 and the sulfamoyloxy group is bound to the aromatic A-ring,

### D Ring Modifications

In one preferred aspect the compound is a compound in accordance with the teachings of WO 03/033518 A typical compound is a compound having the Formula

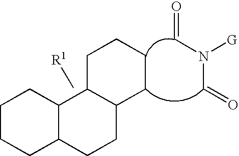

wherein G is H or a substituent, and wherein R¹ is any one of a sulphamate group, a phosphonate group, a thiophosphonate group, a sulphonate group or a sulphonamide group. Such compounds include a compound having the Formula

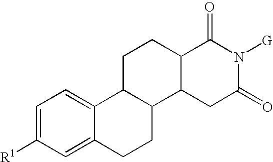

wherein R¹ is a sulphamate group of the formula (R₄)(R₅)NSO₂—O—; R⁴ and R⁵ are independently selected from hydrogen, alkyl, cycloalkyl, alkenyl and aryl or combinations thereof, or together represent alkylene, wherein the or each alkyl or cycloalkyl or alkenyl contain one or more heteroatoms or groups; G is H or a substituent selected from OH or a hydrocarbyl group; wherein the ring system is optionally substituted by one or more substituents selected from hydroxy, alkyl, alkoxy, alkynyl and halogen. Such compounds include compounds having the formula:

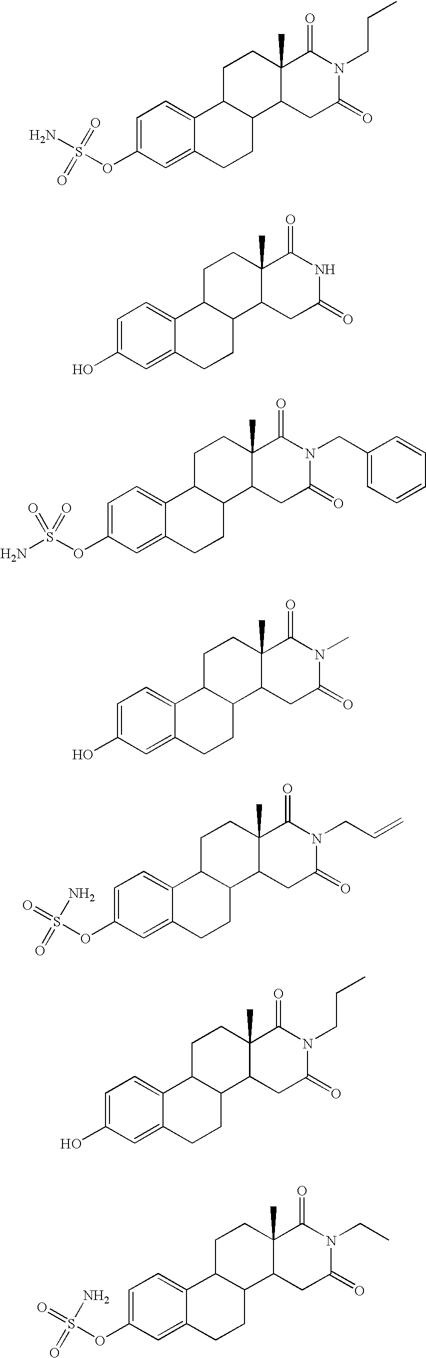

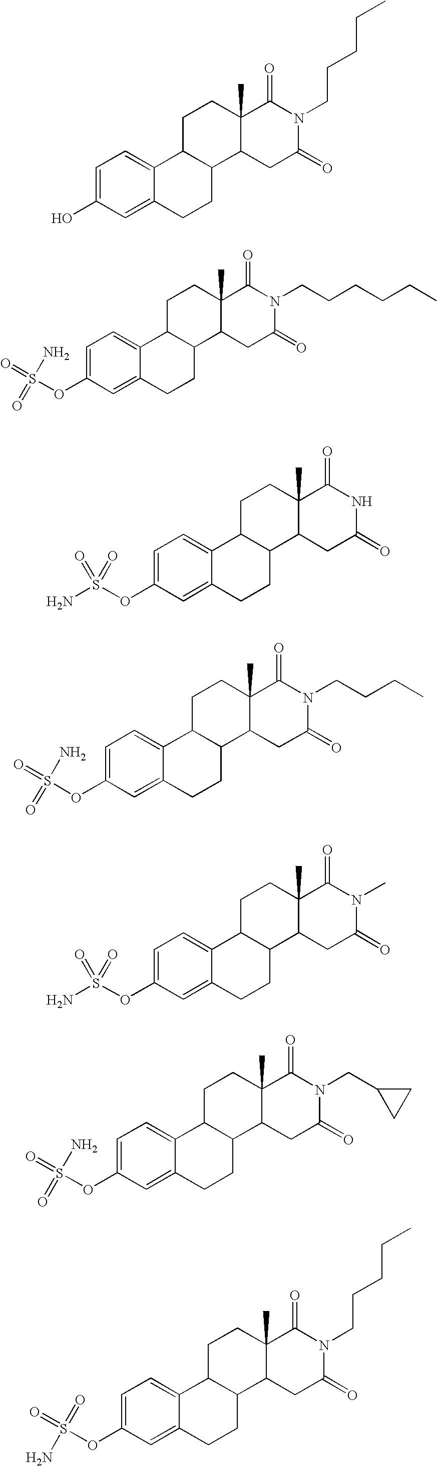

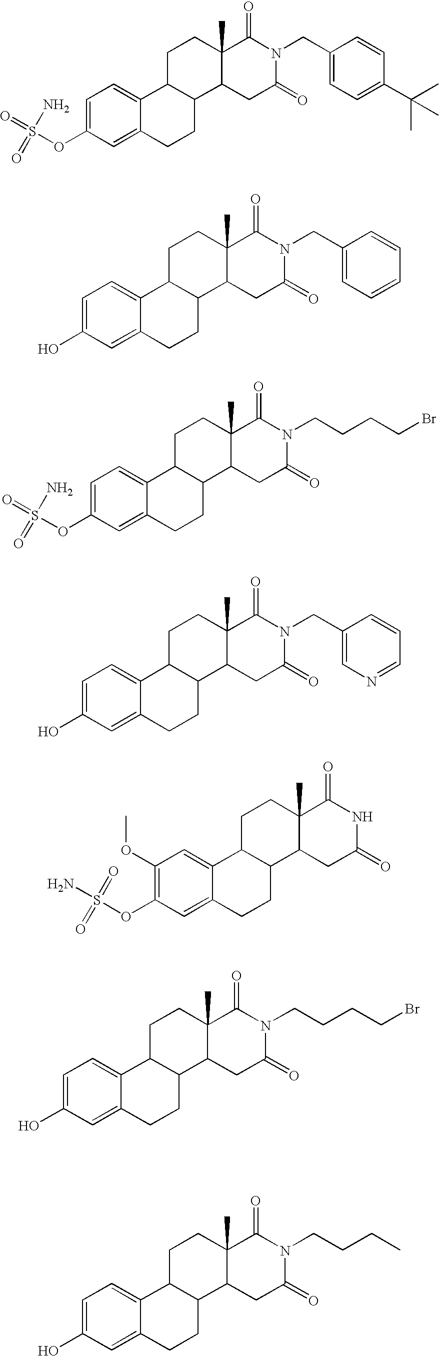

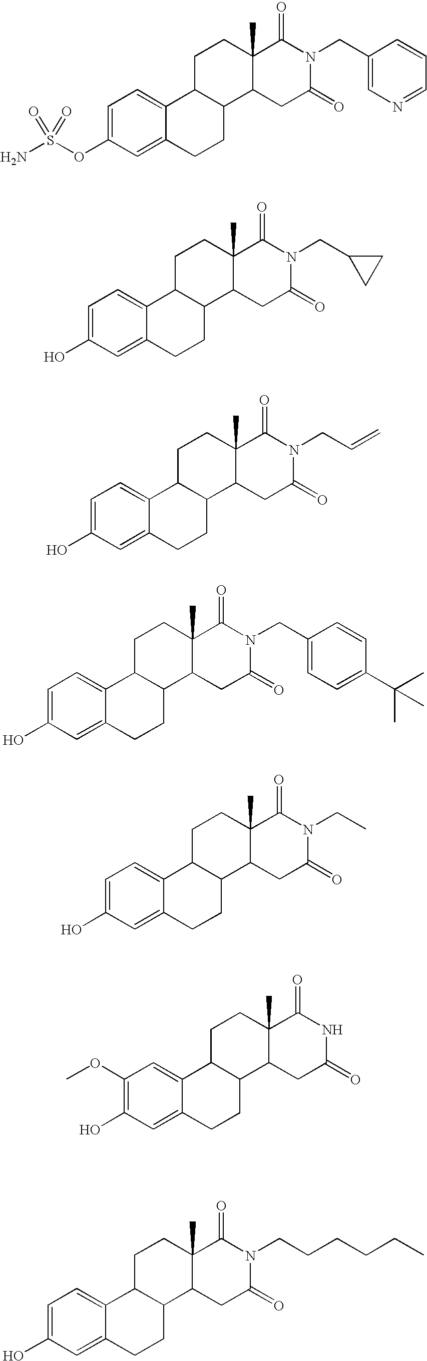

such as compounds of the formula

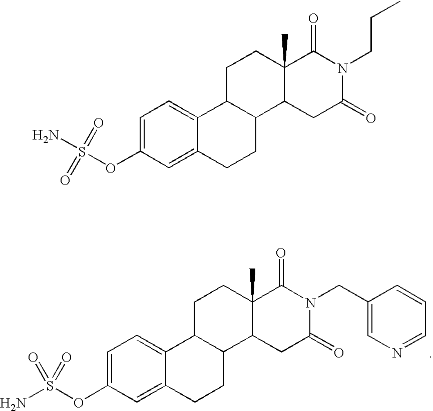

In one preferred aspect the compound is a compound in accordance with the teachings of WO 2004/085459. A typical compound is a compound comprising a steroidal ring system and an optional group R¹ selected from any one of —OH, a sulphamate group, a phosphonate group, a thiophosphonate group, a sulphonate group or a sulphonamide group; wherein the D ring of the steroidal ring system is substituted by a group R² of the formula -L-R³, wherein L is an optional linker group and R³ is selected from groups which are or which comprise one of a nitrile group, an alcohol, an ester, an ether, an amine and an alkene, provided that when R³ is or comprises an alcohol, L is present; and wherein the A ring of the steroidal ring system is substituted at position 2 or 4 with a group R⁴, wherein R⁴ is a hydrocarbyl group.

### Dual Inhibitors

In some aspects the compounds are capable of inhibiting other than steroid sulphatase. For example in one aspect the compound is capable of inhibiting steroid sulphatase and aromatase.

In one preferred aspect the compound is a compound in accordance with the teachings of WO 03/045925. A typical compound is a compound of the formula

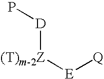

wherein each T is independently selected from H, hydrocarbyl, —F—R, and a bond with one of D, E, P or Q, or together with one of P and Q forms a ring; Z is a suitable atom the valency of which is m; D, E and F are each independently of each other an optional linker group, wherein when Z is nitrogen E is other than CH₂ and C═O; P, Q and R are independently of each other a ring system; and at least Q comprises a sulphamate group.

In one preferred aspect the compound is a compound in accordance with the teachings of one of WO 97/32872, U.S. Pat. No. 6,083,978 and U.S. Pat. No. 6,506,792. A typical compound is a of the general formula

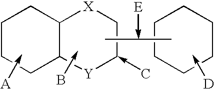

wherein A represents the first ring structure, B represents the third ring structure, D represents the second ring structure, C is an optional double bond, E is a link joining the second ring structure to the third ring structure, X represents a suitable first group, and Y represents a suitable second group; wherein any one of ring structures A, B and D is a phenolic ring; and wherein any one of ring structures A, B and D has bound thereto a sulphamate group. Such compounds include a compound of the general formula

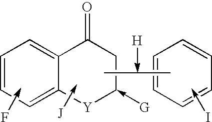

wherein F represents a phenolic ring structure (the first ring structure), J represents the third ring structure, I represents a phenolic ring structure (the second ring structure), G is an optional double bond, H is a link joining the second ring structure to the third ring structure, and Y represents a suitable second group; wherein any one of ring structures F, J and I has bound thereto a sulphamate group. Such compounds include a compound of the general formulae

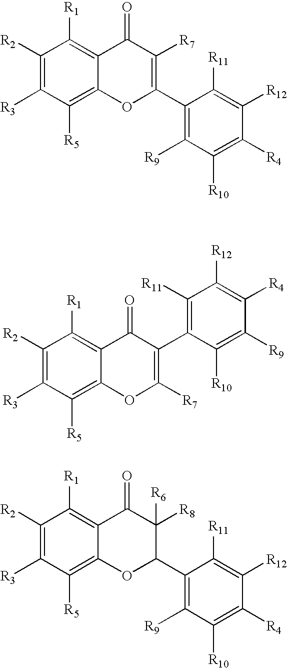

wherein R₁-R₁₂ are independently selected from H, OH, a halogen, an amine, an amide, a sulphonamine, a sulphonamide, any other sulphur containing group, a saturated or unsaturated C₁₋₁₀ alkyl, an aryl group, a saturated or unsaturated C₁₋₁₀ ether, a saturated or unsaturated C₁₋₁₀ ester, a phosphorous containing group; and wherein at least one of R₁-R₁₂ is a sulphamate group.

### Other Steroid Sulphatase Inhibitors

In some aspects the compound is a compound in accordance with the teachings of one of:
Birnböck H, von Angerer E 1990 Sulfate derivatives of 2-phenylindoles as novel steroid sulfatase inhibitors. Biochem Pharmacol 39:1709-1713
Evans T R J, Rowlands M G, Jarman M, Coombes R C 1991 Inhibition of estrone sulfatase enzyme in human placenta and human breast-carcinoma. J Steroid Biochem Mol Biol 39:493-499
Wong C K, Keung W M 1997 Daidzein sulfoconjugates are potent inhibitors of sterol sulfatase (EC 3.1.6.2). Biochem Biophys Res Commun 233:579-583
Anderson C J, Lucas L J H, Widlanski T S 1995 Molecular recognition in biological systems: phosphate esters vs sulfate esters and the mechanism of action of steroid sulfatases. J Am Chem Soc 117:3889-3890
Howarth N M, Purohit A, Reed M J, Potter B V L 1997 Estrone sulfonates as inhibitors of estrone sulfatase. Steroids 62:346-350
Li P-K, Pillai R, Dibbelt L 1995 Estrone sulfate analogs as estrone sulfatase inhibitors. Steroids 60:299-306
Li P-K, Pillai R, Young B L, Bender W H, Martino D M, Lin F T 1993 Synthesis and biochemical studies of estrone sulfatase inhibitors. Steroids 58:106-111
Dibbelt L, Li P-K, Pillai R, Knuppen R 1994 Inhibition of human placental sterylsulfatase by synthetic analogs of estrone sulfate. J Steroid Biochem Mol Biol 50:261-266
Anderson C, Freeman J, Lucas L H, Farley M, Dalhoumi H, Widlanski T S 1997 Estrone sulfatase: probing structural requirements for substrate and inhibitor recognition. Biochem 36:2586-2594
Howarth N M, Purohit A, Reed M J, Potter B V L 1994 Estrone sulfamates: potent inhibitors of estrone sulfatase with therapeutic potential. J Med Chem 37:219-221
Woo L W L, Lightowler M, Purohit A, Reed M J, Potter B V L 1996 Heteroatom-substituted analogues of the active site directed inhibitor estra-1,3,5(10)-trien-17-one-3-sulphamate inhibit estrone sulphatase by a different mechanism. J Steroid Biochem Mol Biol 57:79-88
Selcer K W, Jagannathan S, Rhodes M E, Li P K 1996 Inhibition of placental estrone sulfatase activity and MCF-7 breast cancer cell proliferation by estrone-3-amino derivatives. J Steroid Biochem Mol Biol 59:83-91
Poirier D, Boivin R P 1998 17α-alkyl- or 17-α-substituted benzyl-17β-estradiols: a new family of estrone sulfatase inhibitors. Bioorg Med Chem Lett 8:1891-1896
Boivin R P, Luu-The V, Lachance R, Labrie F, Poirier D 2000 Structure-activity relationships of 17α-derivatives of estradiol as inhibitors of steroid sulfatase. J Med Chem 43:4465-4478
Boivin R P, Labrie F, Poirier D 1999 17α-Alkan (or alkyn) amide derivatives of estradiol as inhibitors of steroid sulfatase activity. Steroids 64:825-833
Ciobanu L C, Boivin R P, Luu-The V, Poirier D 2003 3β-Sulfamate derivatives of C19 and C21 steroids bearing a t-butylbenzyl or a benzyl group: synthesis and evaluation as non-estrogenic and non-androgenic steroid sulfatase inhibitors. J Enz Inhib Med Chem 18:15-26
Chu G H, Peters A, Selcer K W, Li P K 1999 Synthesis and sulfatase inhibitory activities of (E)- and (Z)-4-hydroxytamoxifen sulfamates. Bioorg Med Chem Lett 9:141-144
Golob T, Liebl R, von Angerer E 2002 Sulfamoyloxy-substituted 2-phenylindoles: antiestrogen-based inhibitors of the steroid sulfatase in human breast cancer cells. Bioorg Med Chem Lett:3941-3953
Jütten P, Schumann W, Härtl A, Heinisch L, Gräfe U, Werner W, Ulbricht H 2002 A novel type of nonsteroidal estrone sulfatase inhibitors. Bioorg Med Chem Lett 12:1339-1342
Nussbaumer P, Geyl D, Horvath A, Lehr P, Wolff B, Billich A 2003 Nortropinyl-arylsulfonylureas as novel, reversible inhibitors of human steroid sulfatase. Bioorg Med Chem Lett 13:3673-3677
Lee W, DeRome M, DeBear J, Noell S, Epstein D, Mahle C, DeCarr L, Woodruff K, Huang Z, Dumas J Aryl piperazines: a new class of steroid sulfatase inhibitors for the treatment of hormone-dependent breast cancer. 226^{th} ACS National Meeting, New York, September 2003, poster 301
Carlstrom K, Doberl A, Gershagen S, Rannevik G 1984 Peripheral plasma levels of dehydroepiandrosterone sulphate, dehydroepiandrosterone, androstenedione and testosterone following different doses of danazol. Acta Obstet Gynecol Scand 123 (Suppl.):125-129
Chetrite G, Paris J, Botella J, Pasqualini J R 1996 Effect of nomegestrol acetate on estrone sulfatase and 17β-hydroxysteroid dehydrogenase activities in human breast cancer cells. J Steroid Biochem Mol Biol 58:525-531
Prost-Avallet O, Oursin J, Adessi G L 1991 In vitro effect of synthetic progestogens on estrone sulfatase activity in human breast carcinoma. J Steroid Biochem Mol Biol 39:967-973
Chetrite G, Kloosterboer H J, Pasqualini J R 1997 Effect of tibolone (ORG OD14) and its metabolites on estrone sulphatase activity in MCF-7 and T-47D mammary cancer cells. Anticancer Res 17:135-140
Santner S J, Santen R J 1993 Inhibition of estrone sulfatase and 17β-hydroxysteroid dehydrogenase by antiestrogens. J Steroid Biochem Mol Biol 45:383-390
Zhu B T, Kosh J W, Fu J-H, Cai M X, Xu S, Conney A H 2000 Strong inhibition of estrone-3-sulfatase activity by pregnenolone 16α-carbonitrile but not by several analogs lacking a 16α-nitrile group. Steroids 65:521-527
Horvath, A, Nussbaumer, P, Wolff, B, Billich A 2004 2-(1-Adamantyl)-4-(thio)chromenone-6-carboxylic Acids: Potent Reversible Inhibitors of Human Steroid Sulfatase J. Med. Chem. 47(17): 4268-4276
Lehr P, Billich A, Wolff B, Nussbaumer P 2005 N-Acyl arylsulfonamides as novel, reversible inhibitors of human steroid sulfatase Bioorganic & Medicinal Chemistry Letters, 15: 1235-1238

The compounds of the present invention may comprise other substituents. These other substituents may, for example, further increase the activity of the compounds of the present invention and/or increase stability (ex vivo and/or in vivo).

### Hydrocarbyl Group

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C, then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

The hydrocarbyl/hydrocarbon/alkyl may be straight chain or branched and/or may be saturated or unsaturated.

In one preferred aspect the hydrocarbyl/hydrocarbon/alkyl may be selected from straight or branched hydrocarbon groups containing at least one hetero atom in the group.

In one preferred aspect the hydrocarbyl/hydrocarbon/alkyl may be a hydrocarbyl group comprising at least two carbons or wherein the total number of carbons and hetero atoms is at least two.

In one preferred aspect the hydrocarbyl/hydrocarbon/alkyl may be selected from hydrocarbyl groups containing at least one hetero atom in the group. Preferably the hetero atom is selected from sulphur, nitrogen and oxygen.

In one preferred aspect the hydrocarbyl/hydrocarbon/alkyl may be selected from straight or branched hydrocarbon groups containing at least one hetero atom in the group. Preferably the hetero atom is selected from sulphur, nitrogen and oxygen.

In one preferred aspect the hydrocarbyl/hydrocarbon/alkyl may be selected from straight or branched alkyl groups, preferably C₁₋₁₀ alkyl, more preferably C₁₋₅ alkyl, containing at least one hetero atom in the group. Preferably the hetero atom is selected from sulphur, nitrogen and oxygen.

In one preferred aspect the hydrocarbyl/hydrocarbon/alkyl may be selected from straight chain alkyl groups, preferably C₁₋₁₀ alkyl, more preferably C₁₋₅ alkyl, containing at least one hetero atom in the group. Preferably the hetero atom is selected from sulphur, nitrogen and oxygen.

The hydrocarbyl/hydrocarbon/alkyl may be selected from
C₁-C₁₀ hydrocarbyl,
C₁-C₅ hydrocarbyl
C₁-C₃ hydrocarbyl.
hydrocarbon groups
C₁-C₁₀ hydrocarbon
C₁₀C₅ hydrocarbon
C₁-C₃ hydrocarbon.
alkyl groups
C₁-C₁₀ alkyl
C₁₀C₅ alkyl
C₁-C₃ alkyl.

The hydrocarbyl/hydrocarbon/alkyl may be straight chain or branched and/or may be saturated or unsaturated.

The hydrocarbyl/hydrocarbon/alkyl may be straight or branched hydrocarbon groups containing at least one hetero atom in the group.

### Oxyhydrocarbyl Group

A typical hydrocarbyl group is a oxyhydrocarbyl group.

The term “oxyhydrocarbyl” group as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C, then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Each of the above teachings in respect of hydrocarbyl groups equally applies to the analogous oxyhydrocarbyl groups, that is the corresponding oxyhydrocarbyl group which comprises an oxygen in addition to the hydrocarbyl.

Typically, the oxyhydrocarbyl group is of the formula C₁₋₆O (such as a C₁₋₃O).

### Other Aspects

For some applications, preferably the compounds have no, or a minimal, oestrogenic effect.

For some applications, preferably the compounds have an oestrogenic effect.

For some applications, preferably the compounds have a reversible action.

For some applications, preferably the compounds have an irreversible action.

The present invention also covers novel intermediates that are useful to prepare the compounds of the present invention and metabolites of the compounds of the present invention. For example, the present invention covers novel alcohol precursors for the compounds. By way of further example, the present invention covers bis protected precursors for the compounds. Examples of each of these precursors are presented herein. The present invention also encompasses a process comprising each or both of those precursors for the synthesis of the compounds of the present invention.

In further aspects, the present invention provides
a method of treating cancer, wherein the cancer is of a type in which the cancer cells overexpress aromatase enzyme, comprising administering to a subject a therapeutically effective amount of compound capable of inhibiting a steroid sulphatase enzyme (E.C.3.1.6.2), such that said cancer in said subject is treated.
a method of treating a tumour, wherein the tumour is formed from cancer cells overexpress aromatase enzyme, comprising administering to a subject a therapeutically effective amount of compound capable of inhibiting a steroid sulphatase enzyme (E.C.3.1.6.2), such that said tumour in said subject is treated.
a method of treating a proliferative disease, wherein the proliferative disease is of a type in which the proliferative cells overexpress aromatase enzyme, comprising administering to a subject a therapeutically effective amount of compound capable of inhibiting a steroid sulphatase enzyme (E.C.3.1.6.2), such that said proliferative disease in said subject is treated.
a method of treating cancer associated with overexpressed aromatase enzyme, comprising administering to a subject a therapeutically effective amount of compound capable of inhibiting a steroid sulphatase enzyme (E.C.3.1.6.2), such that said cancer in said subject is treated.
a method of treating a tumour associated with overexpressed aromatase enzyme, comprising administering to a subject a therapeutically effective amount of compound capable of inhibiting a steroid sulphatase enzyme (E.C.3.1.6.2), such that said tumour in said subject is treated.

### Steroid Sulphatase

Steroid sulphatase—which is sometimes referred to as steroid sulphatase or steryl sulphatase or “STS” for short—hydrolyses several sulphated steroids, such as oestrone sulphate, dehydroepiandrosterone sulphate and cholesterol sulphate. STS has been allocated the enzyme number EC 3.1.6.2.

STS has been cloned and expressed. For example see Stein et al (J. Biol. Chem. 264:13865-13872 (1989)) and Yen et al (Cell 49:443-454 (1987)).

STS is an enzyme that has been implicated in a number of disease conditions.

By way of example, workers have found that a total deficiency in STS produces ichthyosis. According to some workers, STS deficiency is fairly prevalent in Japan. The same workers (Sakura et al, J Inherit Metab Dis 1997 November; 20(6):807-10) have also reported that allergic diseases—such as bronchial asthma, allergic rhinitis, or atopic dermatitis—may be associated with a steroid sulphatase deficiency.

In addition to disease states being brought on through a total lack of STS activity, an increased level of STS activity may also bring about disease conditions. By way of example, and as indicated above, there is strong evidence to support a role of STS in breast cancer growth and metastasis.

STS has also been implicated in other disease conditions. By way of example, Le Roy et al (Behav Genet. 1999 March; 29(2):131-6) have determined that there may be a genetic correlation between steroid sulphatase activity and initiation of attack behaviour in mice. The authors conclude that sulphatation of steroids may be the prime mover of a complex network, including genes shown to be implicated in aggression by mutagenesis.

### STS Inhibitor

In accordance with the present invention, the compound of the present invention is capable of acting as an STS inhibitor.

Here, the term “inhibitor” as used herein with respect to the compound of the present invention means a compound that can inhibit STS activity—such as reduce and/or eliminate and/or mask and/or prevent the detrimental action of STS. The STS inhibitor may act as an antagonist.

The ability of compounds to inhibit oestrone sulphatase activity can be assessed using either intact JEG3 choriocarcinoma cells or placental microsomes. In addition, an animal model may be used. Details on suitable Assay Protocols are presented in following sections. It is to be noted that other assays could be used to determine STS activity and thus STS inhibition. For example, reference may also be made to the teachings of WO-A-99/50453.

In one aspect, for some applications, the compound is further characterised by the feature that if the sulphamate group were to be substituted by a sulphate group to form a sulphate derivative, then the sulphate derivative would be hydrolysable by an enzyme having steroid sulphatase (E.C. 3.1.6.2) activity—i.e. when incubated with steroid sulphatase EC 3.1.6.2 at pH 7.4 and 37° C.

In one preferred embodiment, if the sulphamate group of the compound were to be replaced with a sulphate group to form a sulphate compound then that sulphate compound would be hydrolysable by an enzyme having steroid sulphatase (E.C. 3.1.6.2) activity and would yield a Km value of less than 200 mmolar, preferably less than 150 mmolar, preferably less than 100 mmolar, preferably less than 75 mmolar, preferrably less than 50 mmolar, when incubated with steroid sulphatase EC 3.1.6.2 at pH 7.4 and 37° C.

For some applications, preferably the compound of the present invention has at least about a 100 fold selectivity to a desired target (e.g. STS and/or aromatase), preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

It is to be noted that the compound of the present invention may have other beneficial properties in addition to or in the alternative to its ability to inhibit STS and/or aromatase activity.

### Other Substituents

The compound of the present invention may have substituents other than those of shown in the general formulae. By way of example, these other substituents may be one or more of: one or more sulphamate group(s), one or more phosphonate group(s), one or more thiophosphonate group(s), one or more sulphonate group(s), one or more sulphonamide group(s), one or more halo groups, one or more O groups, one or more hydroxy groups, one or more amino groups, one or more sulphur containing group(s), one or more hydrocarbyl group(s)—such as an oxyhydrocarbyl group.

### Assay for Determining STS Activity Using Cancer Cells

### (Protocol 1)

Inhibition of Steroid Sulphatase Activity in JEG3 Cells

Steroid sulphatase activity is measured in vitro using intact JEG3 choriocarcinoma cells. This cell line may be used to study the control of human breast cancer cell growth. It possesses significant steroid sulphatase activity (Boivin et al., J. Med. Chem., 2000, 43: 4465-4478) and is available in from the American Type Culture Collection (ATCC).

Cells are maintained in Minimal Essential Medium (MEM) (Flow Laboratories, Irvine, Scotland) containing 20 mM HEPES, 5% foetal bovine serum, 2 mM glutamine, non-essential amino acids and 0.075% sodium bicarbonate. Up to 30 replicate 25 cm² tissue culture flasks are seeded with approximately 1×10⁵ cells/flask using the above medium. Cells are grown to 80% confluency and the medium is changed every third day.

Intact monolayers of JEG3 cells in triplicate 25 cm² tissue culture flasks are washed with Earle's Balanced Salt Solution (EBSS from ICN Flow, High Wycombe, U.K.) and incubated for 3-4 hours at 37° C. with 5 pmol (7×10⁵ dpm) [6, 7-3H]oestrone-3-sulphate (specific activity 60 Ci/mmol from New England Nuclear, Boston, Mass., U.S.A.) in serum-free MEM (2.5 ml) together with oestrone-3-sulphamate (11 concentrations: 0; 1 fM; 0.01 pM; 0.1 pM; 1 pM; 0.01 nM; 0.1 nM; 1 nM; 0.01 mM; 0.1 mM; 1 mM). After incubation each flask is cooled and the medium (1 ml) is pipetted into separate tubes containing [14C]oestrone (7×10³ dpm) (specific activity 50 Ci/mmol from Amersham International Radiochemical Centre, Amersham, U.K.). The mixture is shaken thoroughly for 30 seconds with toluene (5 ml). Experiments have shown that >90% [14C]oestrone and <0.1% [3H]oestrone-3-sulphate is removed from the aqueous phase by this treatment. A portion (2 ml) of the organic phase is removed, evaporated and the 3H and 14C content of the residue determined by scintillation spectrometry. The mass of oestrone-3-sulphate hydrolysed was calculated from the 3H counts obtained (corrected for the volumes of the medium and organic phase used, and for recovery of [14C]oestrone added) and the specific activity of the substrate. Each batch of experiments includes incubations of microsomes prepared from a sulphatase-positive human placenta (positive control) and flasks without cells (to assess apparent non-enzymatic hydrolysis of the substrate). The number of cell nuclei per flask is determined using a Coulter Counter after treating the cell monolayers with Zaponin. One flask in each batch is used to assess cell membrane status and viability using the Trypan Blue exclusion method (Phillips, H. J. (1973) In: Tissue culture and applications, [eds: Kruse, D. F. & Patterson, M. K.]; pp. 406-408; Academic Press, New York).

Results for steroid sulphatase activity are expressed as the mean±1 S.D. of the total product (oestrone+oestradiol) formed during the incubation period (3-4 hours) calculated for 106 cells and, for values showing statistical significance, as a percentage reduction (inhibition) over incubations containing no oestrone-3-sulphamate. Unpaired Student's t-test was used to test the statistical significance of results.

### Assay for Determining STS Activity Using Placental Microsomes

### (Protocol 2)

Inhibition of Steroid Sulphatase Activity in Placental Microsomes

Sulphatase-positive human placenta from normal term pregnancies are thoroughly minced with scissors and washed once with cold phosphate buffer (pH 7.4, 50 mM) then re-suspended in cold phosphate buffer (5 ml/g tissue). Homogenisation is accomplished with an Ultra-Turrax homogeniser, using three 10 second bursts separated by 2 minute cooling periods in ice. Nuclei and cell debris are removed by centrifuging (4° C.) at 2000 g for 30 minutes and portions (2 ml) of the supernatant are stored at 20° C. The protein concentration of the supernatants is determined by the method of Bradford (Anal. Biochem., 72, 248-254 (1976)).

Incubations (1 ml) are carried out using a protein concentration of 100 μg/ml, substrate concentration of 20 μM [6,7-3H]oestrone-3-sulphate (specific activity 60 Ci/mmol from New England Nuclear, Boston, Mass., U.S.A.) and an incubation time of 20 minutes at 37° C. If necessary eight concentrations of compounds are employed: 0 (i.e. control); 0.05 μM; 0.1 μM; 0.2 μM; 0.4 μM; 0.6 μM; 0.8 μM; 1.0 μM, 10.0 μM. After incubation each sample is cooled and the medium (1 ml) was pipetted into separate tubes containing [14C]oestrone (7×10³ dpm) (specific activity 50 Ci/mmol from Amersham International Radiochemical Centre, Amersham, U.K.). The mixture is shaken thoroughly for 30 seconds with toluene (5 ml). Experiments have shown that >90% [14C]oestrone and <0.1% [3H]oestrone-3-sulphate is removed from the aqueous phase by this treatment. A portion (2 ml) of the organic phase was removed, evaporated and the 3H and 14C content of the residue determined by scintillation spectrometry. The mass of oestrone-3-sulphate hydrolysed is calculated from the 3H counts obtained (corrected for the volumes of the medium and organic phase used, and for recovery of [14C]oestrone added) and the specific activity of the substrate.

### Animal Assay Model for Determining STS Activity

### (Protocol 3)

Inhibition of Oestrone Sulphatase Activity In Vivo

The compounds of the present invention may be studied using an animal model, in particular in ovariectomised rats. In this model compounds which are oestrogenic stimulate uterine growth.

The compound (0.1-10 mg/Kg/day for five days) is administered orally to rats with another group of animals receiving vehicle only (propylene glycol). At the end of the study samples of liver tissue were obtained and oestrone sulphatase activity assayed using 3H oestrone sulphate as the substrate as previously described (see PCT/GB95/02638).

### Animal Assay Model for Determining Oestrogenic Activity

### (Protocol 4)

The compounds of the present invention may be studied using an animal model, in particular in ovariectomised rats. In this model, compounds which are oestrogenic stimulate uterine growth.

The compound (0.1-10 mg/Kg/day for five days) was administered orally to rats with another group of animals receiving vehicle only (propylene glycol). At the end of the study uteri were obtained and weighed with the results being expressed as uterine weight/whole body weight×100.

Compounds having no significant effect on uterine growth are not oestrogenic.

### Biotechnological Assays for Determining STS Activity

### (Protocol 5)

The ability of compounds to inhibit oestrone sulphatase activity can also be assessed using amino acid sequences or nucleotide sequences encoding STS, or active fragments, derivatives, homologues or variants thereof in, for example, high-through put screens. Such assays and methods for their practice are taught in WO 03/045925 which is incorporated herein by reference.

In one preferred aspect, the present invention relates to a method of identifying agents that selectively modulate STS, which compounds have the formula (I).

### Assay for Determining Aromatase Activity Using JEG3 Cells

### (Protocol 6)

Aromatase activity is measured in JEG3 choriocarcinoma cells, obtained from the ATCC. This cell line possesses significant aromatase activity and is widely used to study the control of human aromatase activity (Bhatnager et al., J. Steroid Biochem. Molec. Biol. 2001, 76: 199-202). Cells are maintained in Minimal Essential Medium (MEM, Flow Laboratories, Irvine, Scotland) containing 20 mM HEPES, 10% foetal bovine serum, 2 mM glutamine, non-essential amino acids and 0.075% sodium bicarbonate. Intact monolayers of JEG3 cells (2.5×10⁶ cells) in triplicate 25 cm² tissue culture flasks are washed with Earle's Balanced salt solution (EBSS, from ICN Flow, High Wycombe, UK) and incubated with [1β-³H]androstenedione (2-5 nM, 26 Ci/mmol, New England Nuclear, Boston, Mass., USA) for 30 min with inhibitors over the range of 10 pm-10 μM. During the aromatase reaction, ³H₂O is liberated which can be quantified using a liquid scintillation spectrometer (Beckman-Coulter, High Wycombe, Bucks. UK). This ³H₂O-release method has been widely used to measure aromatase activity Newton et al., J. Steroid Biochem. 1986,24: 1033-1039). The number of cell nuclei per flask is determined using a Coulter Counter after treating the cell monolayers with Z aponin.

Results for aromatase activity are expressed as the mean±1 S.D. of the product formed during the incubation period (30 min) calculated for 10⁶ cells and, for values showing a statistical significance, as a percentage reduction (inhibition) over incubations containing no aromatase inhibitor. Unpaired Student's t test was used to test the statistical significance of results. IC₅₀ values were calculated as the concentration of inhibitor required to obtain a 50% inhibition of aromatase activity.

### Therapy

As discussed herein in one aspect the present invention provides use of a compound capable of inhibiting a steroid sulphatase enzyme (E.C.3.1.6.2) in the manufacture of a medicament for the treatment of cancer, wherein the cancer is of a type in which the cancer cells overexpress aromatase enzyme.

The term “treatment” includes curative effects, alleviation effects, and prophylactic effects.

The treatment may be of humans or animals, preferably female humans or animals, preferably female humans.

### Pharmaceutical Compositions

In one aspect, the present invention provides use of a pharmaceutical composition, which comprises a compound as defined herein and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Combination Pharmaceutical

The steroid sulphatase inhibiting compound may be used in combination with one or more other active agents, such as one or more other pharmaceutically active agents.

By way of example, the steroid sulphatase inhibiting compounds may be used in combination with other STS inhibitors and/or other inhibitors such as an aromatase inhibitor (such as for example, letrozole, anastrozole, exemestane, 4-hydroxyandrostenedione (4-OHA)) and/or steroids—such as the naturally occurring neurosteroids dehydroepiandrosterone sulfate (DHEAS) and pregnenolone sulfate (PS) and/or other structurally similar organic compounds. Examples of other STS inhibitors may be found in the above references. By way of example, STS inhibitors for use in the present invention include EMATE, and either or both of the 2-ethyl and 2-methoxy 17-deoxy compounds that are analogous to compound 5 presented herein.

In addition, or in the alternative, the steroid sulphatase inhibiting compound may be used in combination with a biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)— such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions for use in the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.01 to 10 mg/kg body weight, such as from 0.01 to 2 mg/kg body weight, such as from 0.05 to 2 mg/kg body weight, such as from 0.01 to 1 mg/kg body weight, such as from 0.05 to 0.5 mg/kg body weight, such as from 0.05 to 0.3 mg/kg body weight, such as from 0.07 to 0.3 mg/kg body weight.

By way of further example, the steroid sulphatase inhibiting compounds may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Thus, for pharmaceutical administration, the steroid sulphatase inhibiting compounds can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70 Kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

### Compound Preparation

The steroid sulphatase inhibiting compounds may be prepared by reacting an appropriate alcohol with a suitable chloride. By way of example, the sulphamate compounds of the present invention may be prepared by reacting an appropriate alcohol with a suitable sulfamoyl chloride, of the formula R⁷R⁸NSO₂Cl.

Typical conditions for carrying out the reaction are as follows.

Sodium hydride and a sulfamoyl chloride are added to a stirred solution of the alcohol in anhydrous dimethyl formamide at 0° C. Subsequently, the reaction is allowed to warm to room temperature whereupon stirring is continued for a further 24 hours. The reaction mixture is poured onto a cold saturated solution of sodium bicarbonate and the resulting aqueous phase is extracted with dichloromethane. The combined organic extracts are dried over anhydrous MgSO₄. Filtration followed by solvent evaporation in vacuo and co-evaporated with toluene affords a crude residue which is further purified by flash chromatography.

Preferably, the alcohol is derivatised, as appropriate, prior to reaction with the sulfamoyl chloride. Where necessary, functional groups in the alcohol may be protected in known manner and the protecting group or groups removed at the end of the reaction.

Preferably, the sulphamate compounds are prepared according to the teachings of Page et al (1990 Tetrahedron 46; 2059-2068).

The phosphonate compounds may be prepared by suitably combining the teachings of Page et al (1990 Tetrahedron 46; 2059-2068) and PCT/GB92/01586.

The sulphonate compounds may be prepared by suitably adapting the teachings of Page et al (1990 Tetrahedron 46; 2059-2068) and PCT/GB92/01586.

The thiophosphonate compounds may be prepared by suitably adapting the teachings of Page et al (1990 Tetrahedron 46; 2059-2068) and PCT/GB91/00270.

Preferred preparations are also presented in the following text.

The invention will now be further described by way of the following non-limiting examples.

### EXAMPLE

The present invention will now be described in further detail by way of example only with reference to the accompanying figure in which:—

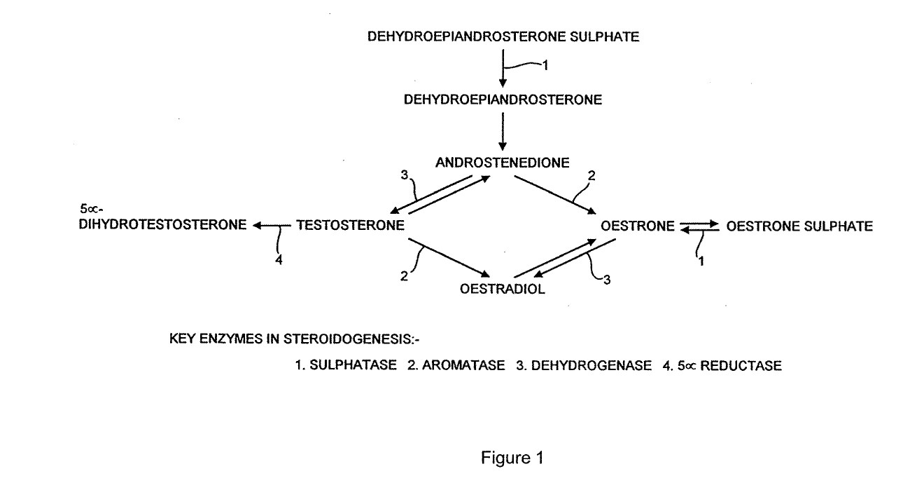

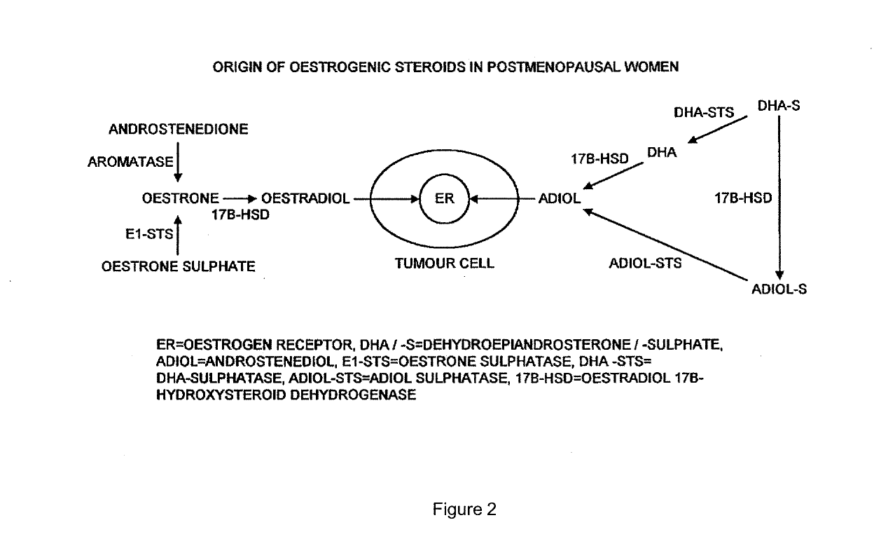

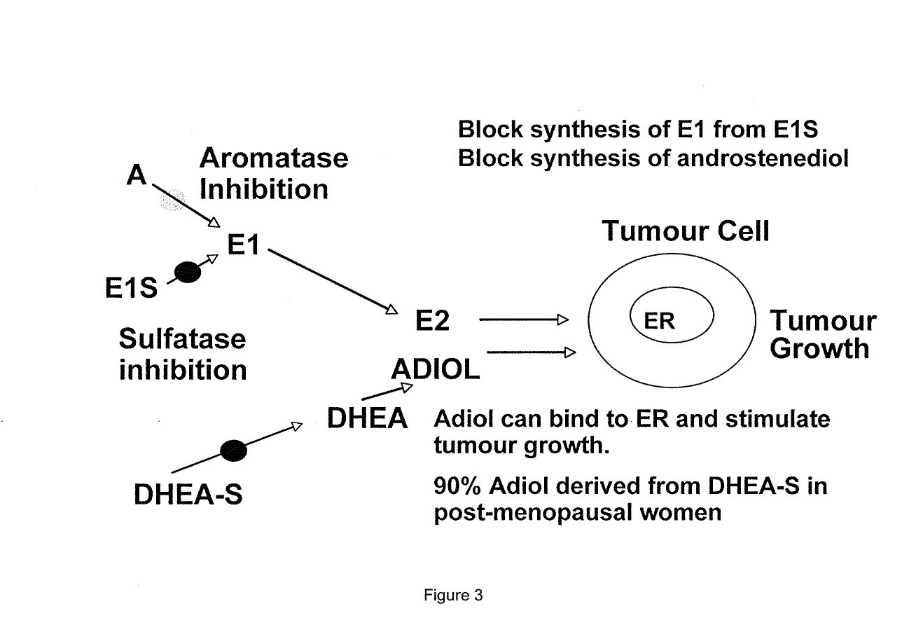

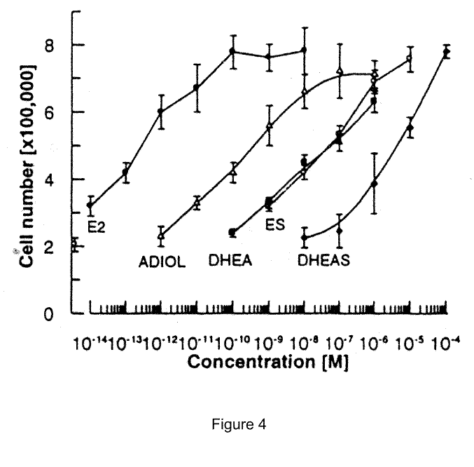

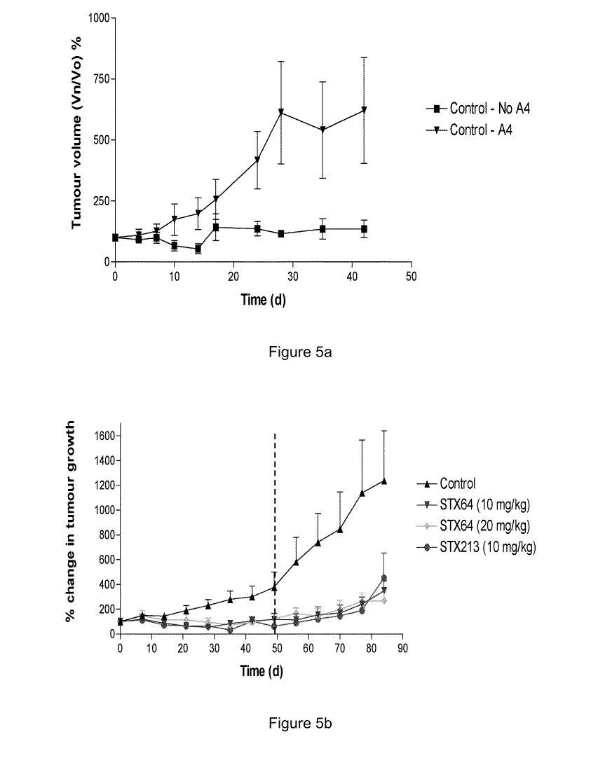

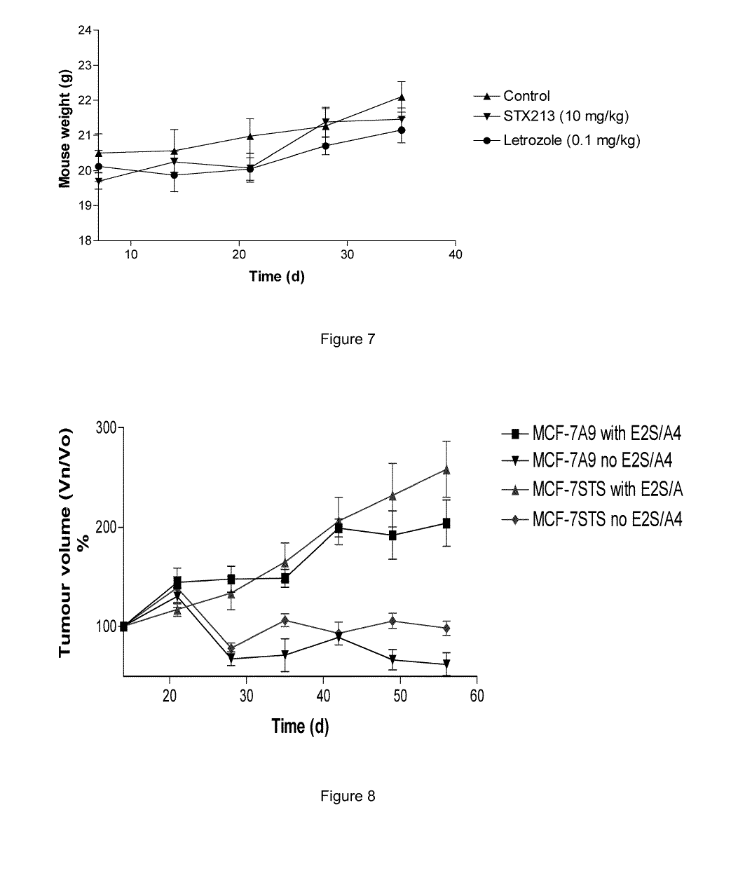

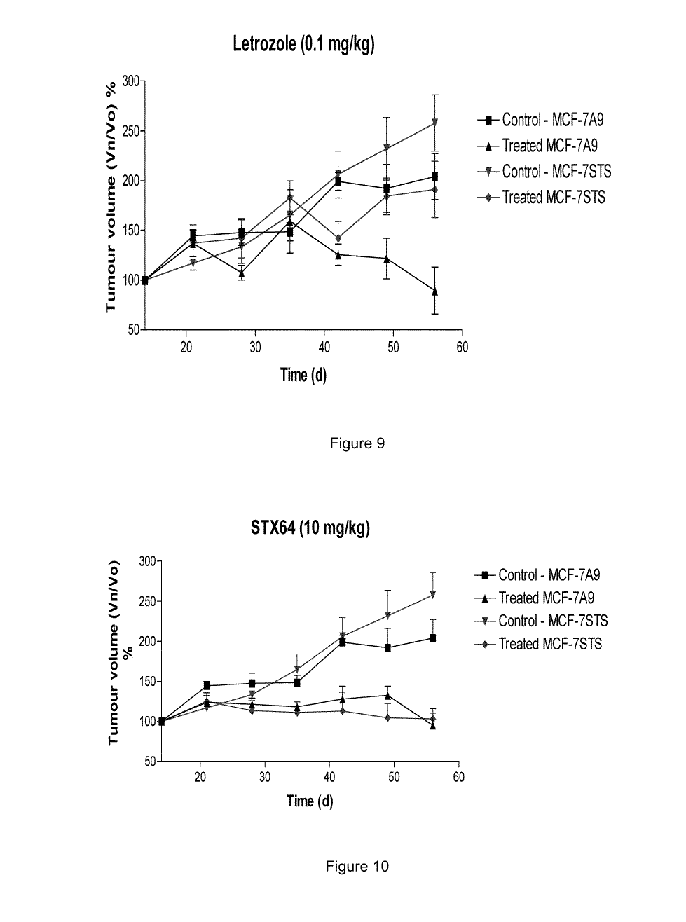

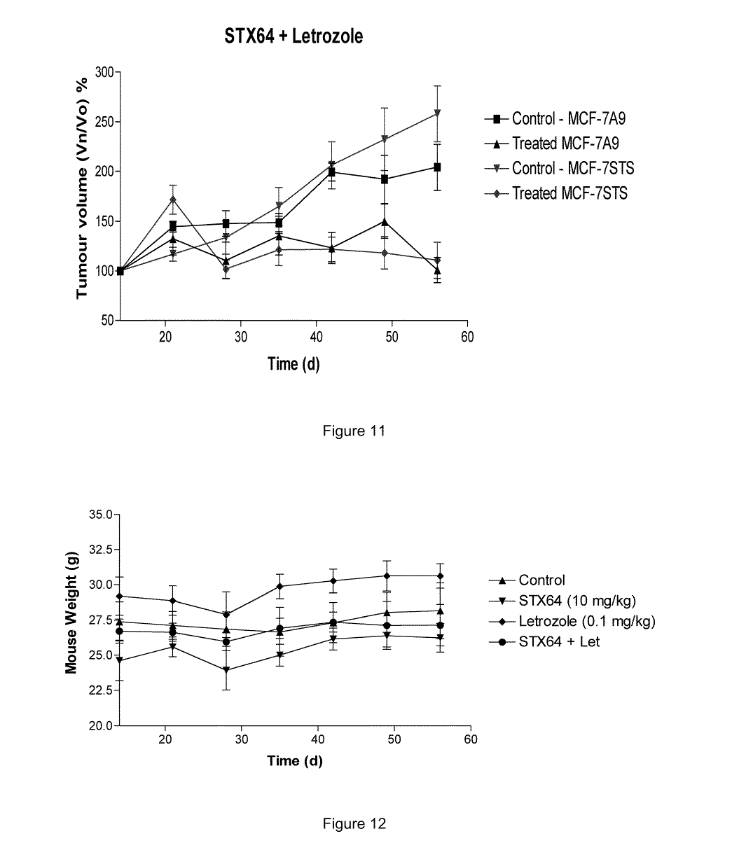

FIG. 1 shows a scheme,
FIG. 2 shows a scheme,
FIG. 3 shows a scheme,
FIG. 4 shows a graph,
FIG. 5 shows a graph,
FIG. 7 shows a graph,
FIG. 8 shows a graph,
FIG. 9 shows a graph,
FIG. 10 shows a graph,
FIG. 11 shows a graph; and
FIG. 12 shows a graph.

The present invention will now be described only by way of example. However, it is to be understood that the examples also present preferred compounds of the present invention, as well as preferred routes for making same and useful intermediates in the preparation of same.

### Compound Preparation

Compound STX 64 (shown below) was prepared in accordance with the teachings of WO 97/30041.

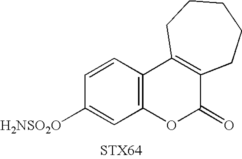

Compound STX 213 (shown below) was prepared in accordance with the teachings of WO 03/033518.

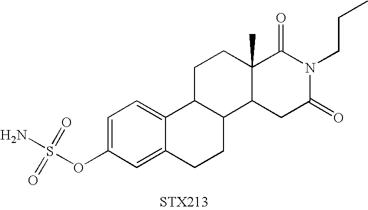

### Biological Data

The assay for the determination of androstenedione, testosterone, E1 and E2 was the gas chromatographic tandem mass spectroscopic method of Wang et al., (2005). Recombinant cell ultra-sensitive bioassay for measurements of estrogens in postmenopausal women J Clin Endocrinol Metab 90: 1407-1413, 2005.

### Background

In addition to the aromatase pathway for the synthesis of estrogens in postmenopausal women, the sulfatase route is also important of the formation of steroids with potent estrogenic properties. Steroid sulfatase (STS) is responsible for the hydrolysis of estrone sulfate (E1S) and dehydroepiandrosterone sulfate (DHEAS) to estrone (E1) and DHEA respectively, which can be reduced in the body to estradiol (E2) and androstenediol (Adiol), both of which have potent estrogenic properties (FIGS. 3 & 4; reviewed in Reed et al., Endocrine Reviews, 256:171-202, 2005). FIG. 4 shows that androgen stimulated growth is blocked by an anti-oestrogen and that an aromatase inhibitor failed to block DHEAS stimulated growth whereas a steroid sulphatase inhibitor did. This provides evidence for an aromatase independent pathway. Results from a recent phase I trial with STX 64 showed that this drug effectively blocked STS activity in peripheral and tumour tissues in postmenopausal women with breast cancer. In addition to reducing serum oestrogen concentrations the drug also, unexpectedly, reduced levels of androstenedione and testosterone, the substrates for the aromatase. This finding indicates that in postmenopausal women androstenedione originates mainly from the peripheral conversion of DHEAS, rather than by direct secretion from the adrenal cortex.

To investigate the relative importance of the aromatase and sulfatase pathways we are developing animal models in which MCF-7 cells stably transfected with the aromatase or STS cDNAs are inoculated into nude mice.

### Pre-Clinical Models

### Mice

Ovariectomised, athymic female MF-1 nude mice (nu/nu) (age 6-8 weeks) were obtained from Harlan Olac. Twenty four hours before the inoculation of MCF-7 cells animals were injected s.c. with androstenedione (A4) or estradiol sulfate (E2S). On the day of inoculation MCF-7 cells (50 μl in Matrigel) were injected s.c. into the flanks of mice. After cell inoculation mice were injected with A4 and E2S and received another injection of these steroids 24 h later. Mice then received A4 plus E2S 3 times per week until the end of the study. When tumours had reached approximately 80 mm³ dosing was initiated with compounds being administered orally (100 μl; vehicle 10% THF: 90% propylene glycol). Tumour measurements and the weight of animals were recorded weekly.

### MCF-7 Cells

MCF-7 cells were routinely cultured in RPMI with 10% FCS. The cDNAs for either the aromatase or STS were cloned into the pCl-Neo vector which contains the neomycin resistant gene and transfected into MCF-7 cells. Stable clones were selected using G418 and cell lines established and evaluated for enzyme expression and activity.

### Model Development

Initial experiments were carried out in which cells over-expressing aromatase (MCF-7_{AROM}) or STS (MCF-7_{STS}) were inoculated into the flanks of different groups of mice. Results from these experiments showed that tumours did not grow in the absence of substrates and that the growth of tumours derived from MCF 7_{STS} cells could be inhibited by STX64 or a second generation STS inhibitor, STX213 (FIG. 5). FIG. 5a shows data in respect of MCF-7 cells transfected with aromatase and the growth of which is stimulated with androstenedione (A4). FIG. 5b shows data in respect of MCF-7 cells transfected with steroid sulphatase (STS) and the growth of which is stimulated with E1S.

### Study 1: MCF-7_{STS}+MCF-7_{AROM} vs MCF-7_{WT}

For this study MCF-7 cells over-expressing aromatase or STS were mixed and inoculated (5×10⁶ cells) in Matrigel into the flanks of nude mice. A similar number of MCF-7_{WT} cells were inoculated into the other flank. For this study growth of tumours was stimulated by s.c. injection of A4 (50 μg) and E2S (50 μg) in 50 μl vehicle.

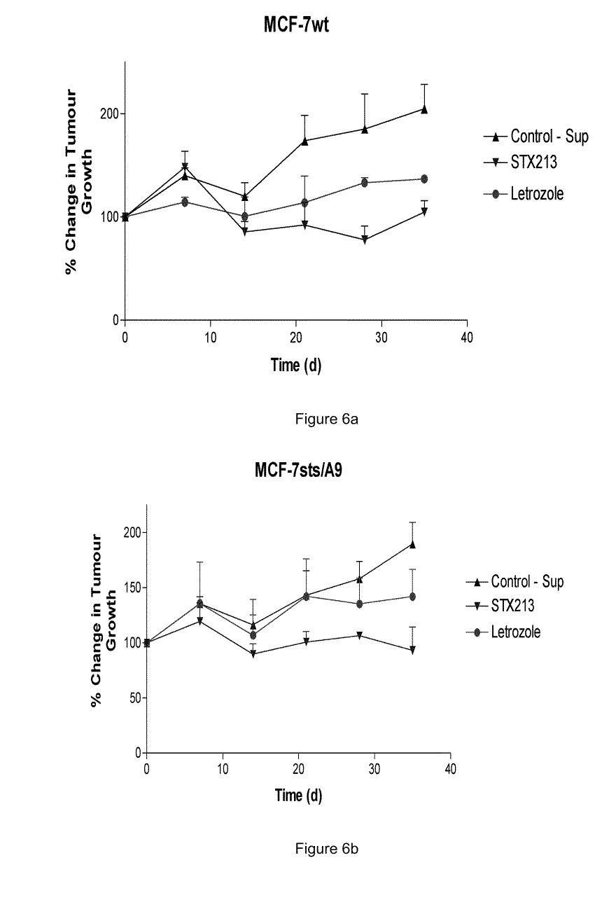

Tumours derived from MCF-7_{WT} and MCF-7_{STS}+MCF-7_{AROM} grew in response to dosing with A4 plus E2S. While oral administration of letrozole (0.1 mg/kg) resulted in some reduction of tumour growth, the second generation STS inhibitor, STX213, significantly reduced the growth of MCF-7_{WT} and MCF-7_{STS}+MCF-7_{AROM} tumours (FIG. 6). Neither STX213 nor letrozole appeared to have any toxic effects, as animals continued to grow throughout the duration of the study (FIG. 7).

FIG. 6a shows that STX213 (a 2nd generation STS inhibitor) inhibited growth of MCF-7wt and tumors derived from MCF-7AROM and MCF-7STS to a greater extent than Letrozole.

FIG. 6b provides data for Letrozole 0.1 mg/kg p.o. and STX213 10 mg/kg p.o. ( 5/7 per week).

FIG. 7 shows that, at the dose tested, STX213 was devoid of any toxicity as shown by its lack of effect on body weight.

### Study 2: MCF-7_{STS} vs MCF-7_{AROM}

This study was similar to the previous one with the exception that MCF-7_{STS} or MCF-7_{AROM} cells (1×10⁷) were inoculated into different flanks of the same animals. The doses of A4 and E2S were increased to 100 μg for each compound.

Tumours derived from MCF-7_{STS} or MCF-7_{AROM} cells grew in the presence of A4 plus E2S but no growth occurred in their absence (FIG. 8). This finding demonstrates the absolute requirement of estrogens derived from A4 and E2S to support tumour growth in this model.

Oral administration of letrozole (0.1 mg/kg) resulted in significant inhibition in the growth of tumours derived from MCF 7_{AROM} cells but did not affect the growth of tumours derived from MCF-7_{STS} cells (FIG. 9).

Oral administration of the STS inhibitor STX64 resulted in significant inhibition of tumour growth derived from not only from MCF-7_{STS} cells, as expected, but also MCF-7_{AROM} cells (FIG. 10). Dosing with the combination of letrozole plus STX64 did not improve the tumour growth inhibition achieved with STX64 alone (FIG. 11). STX64 or letrozole, alone or in combination, were well tolerated with no effects on animal weight being detected (FIG. 12).

### Summary

Results from these preliminary studies have indicated that using a mixed population of MCF-7_{STS} plus MCF-7_{AROM} cells, STX213 appeared to inhibit tumour growth to a greater extent than letrozole. When MCF-7_{STS} or MCF-7_{AROM} cells were inoculated into different flanks of the same animals, while letrozole significantly inhibited the growth of tumours derived from MCF 7_{AROM} cells it had no effect on the growth of tumours derived from MCF-7_{STS} cells. In contrast, STX64 inhibited the growth of both tumour types.

A possible explanation for the ability of STX64 to inhibit the growth of tumours derived from both cell types. After the aromatisation of A4 to E1 it is possible that it is rapidly sulfated to E1S by sulfotransferase enzymes which are widely distributed in mouse tissues. Back conversion of E1S to E1 may be required to simulate growth of the tumours derived from the MCF-7_{AROM} cells but this reaction would be blocked by STX64.

Further studies to examine the relative importance of the aromatase and sulfatase pathways are currently ongoing.

All publications and patents and patent applications mentioned in the above specification are herein incorporated by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

The invention is further described by the following numbered paragraphs:

1. Use of a compound capable of inhibiting a steroid sulphatase enzyme (E.C.3.1.6.2) in the manufacture of a medicament for the treatment of cancer, wherein the cancer is of a type in which the cancer cells overexpress aromatase enzyme.
2. Use according to paragraph 1 wherein the cancer is selected from breast cancer, ovarian cancer, prostate cancer and endometrial cancer.
3. Use according to paragraphs 1 or 2 wherein the cancer is breast cancer.
4. Use according to paragraph 1, 2 or 3 wherein the cancer is hormone dependent.
5. Use according to any one of paragraphs 1 to 4 wherein the cancer is oestrogen dependent.
6. Use according to any one of paragraphs 1 to 5 wherein the cancer is oestrogen dependent breast cancer.
7. Use according to any one of the preceding paragraphs wherein the compound comprises a sulphamate group
8. Use according to any one of the preceding paragraphs wherein compound is of Formula (A),

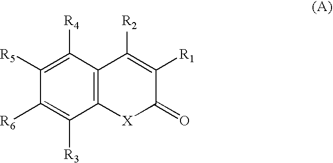

wherein R₁-R₆ are independently selected from H, halo, hydroxy, sulphamate, alkyl and substituted variants or salts thereof; but wherein at least one of R₁-R₆ is a sulphamate group and wherein X is selected from O, NR₉, and CR₁₀R₁₁, wherein R₉ is selected from H and hydrocarbyl, and wherein R₁₀ and R₁₁ are independently selected from H, halo, hydroxy and hydrocarbyl.
9. Use according to paragraph 8 wherein two or more of R₁-R₆ are linked together to form an additional cyclic structure.
10. Use according to paragraphs 8 or 9 wherein X is O.
11. Use according to paragraph 8, 9 or 10, wherein R₁-R₆ are independently selected from H, alkyl and haloalkyl.
12. Use according to paragraph 11 wherein R₁-R₆ are independently selected from H, C₁₋₆ alkyl and C₁₋₆ haloalkyl.
13. Use according to paragraph 11, wherein R₁-R₆ are independently selected from H, C₁₋₃ alkyl and C₁₋₃ haloalkyl.
14. Use according to paragraph 11, wherein R₁-R₆ are independently selected from H, methyl and halomethyl.
15. Use according to any one of the preceding paragraphs, wherein the compound is of Formula (C),

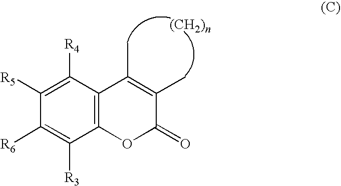

wherein R₃-R₆ are independently selected from H, halo, hydroxy, sulphamate, alkyl and substituted variants or salts thereof; but wherein at least one of R₃-R₆ is a sulphamate group, and wherein n is from 3 to 14.
16. Use according to paragraph 15 wherein n is from 3 to 10.
17. Use according to paragraph 15 wherein n is 5.
18. Use according to any one of paragraphs 8 to 17, wherein R₆ is a sulphamate group.
19. Use according to any one of the preceding paragraphs, wherein the compound is selected from compounds of the Formulae,

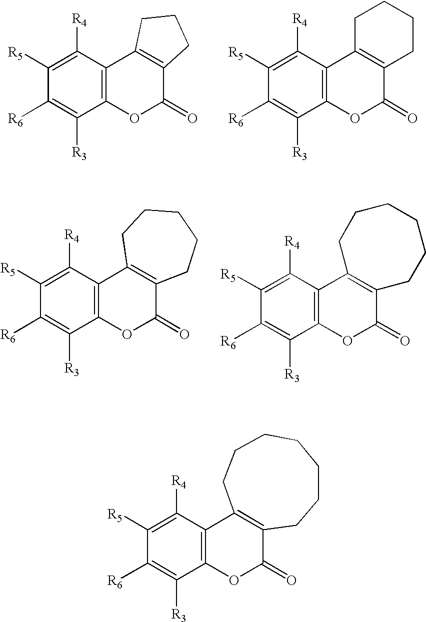

wherein R₃-R₆ are independently selected from H, halo, hydroxy, sulphamate, alkyl and substituted variants or salts thereof; but wherein at least one of R₃-R₆ is a sulphamate group.
20. Use according to any one of paragraphs 7 to 19, wherein the sulphamate group has the formula:

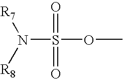

wherein R₇ and R₈ are independently selected from H, alkyl, cycloalkyl, alkenyl, acyl and aryl, or combinations thereof, or together represent alkylene, wherein the or each alkyl or cycloalkyl or alkenyl or optionally contain one or more hetero atoms or groups.
21. Use according to paragraph 20 wherein at least one of R₇ and R₈ is H.
22. Use according to paragraph 20 wherein each of R₇ and R₈ is H.
23. Use according to any one of paragraphs 1 to 7, wherein the compound is selected from compounds of the Formulae

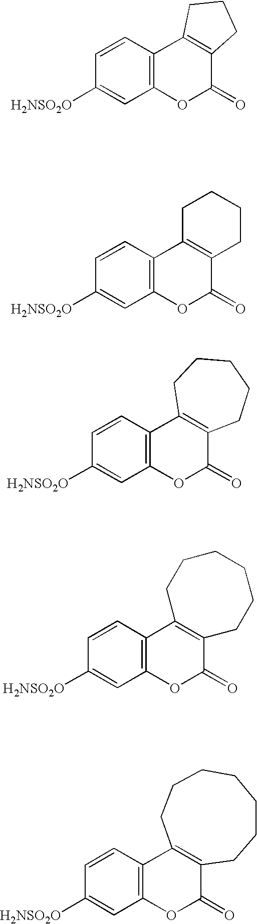

24. Use according to any one of paragraphs 1 to 7, wherein the compound is

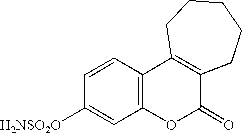

25. A use as substantially hereinbefore described with reference to the Examples.

Having thus descried in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for inhibiting growth of cancer cells comprising administering a compound capable of inhibiting a steroid sulphatase enzyme (E.C.3.1.6.2) to the cancer cells, wherein the cancer is of a type in which the cancer cells overexpress aromatase enzyme.

2. The method according to claim 1 wherein the cancer is selected from breast cancer, ovarian cancer, prostate cancer and endometrial cancer.

3. The method according to claim 2 wherein the cancer is breast cancer.

4. The method according to claim 1 wherein the cancer is hormone dependent.

5. The method according to claim 1 wherein the cancer is oestrogen dependent.

6. The method according to claim 5 wherein the cancer is oestrogen dependent breast cancer.

7. The method according to claim 1 wherein the compound comprises a sulphamate group.

8. The method according to claim 1 wherein compound is of Formula (A), 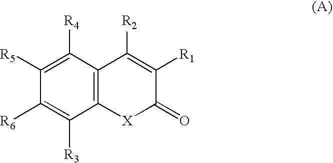
wherein R₁-R₆ are independently selected from H, halo, hydroxy, sulphamate, alkyl and substituted variants or salts thereof; but wherein at least one of R₁-R₆ is a sulphamate group and wherein X is selected from O, NR₉, and CR₁₀R₁₁, wherein R₉ is selected from H and hydrocarbyl, and wherein R₁₀ and R₁₁ are independently selected from H, halo, hydroxy and hydrocarbyl.

9. The method according to claim 8 wherein two or more of R₁-R₆ are linked together to form an additional cyclic structure.

10. The method according to claims 8 wherein X is O.

11. The method according to claim 8 wherein R₁-R₆ are independently selected from H, alkyl and haloalkyl.

12. The method according to claim 11 wherein R₁-R₆ are independently selected from H, C₁₋₆ alkyl and C₁₋₆ haloalkyl.

13. The method according to claim 11, wherein R₁-R₆ are independently selected from H, C₁₋₃ alkyl and C₁₋₃ haloalkyl.

14. The method according to claim 11, wherein R₁-R₆ are independently selected from H, methyl and halomethyl.

15. The method according to claim 1, wherein the compound is of Formula (C), 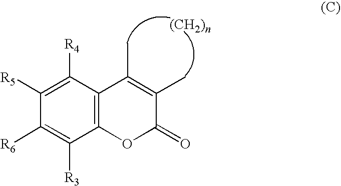
wherein R₃-R₆ are independently selected from H, halo, hydroxy, sulphamate, alkyl and substituted variants or salts thereof; but wherein at least one of R₃-R₆ is a sulphamate group, and wherein n is from 3 to 14.

16. The method according to claim 15 wherein n is from 3 to 10.

17. The method according to claim 15 wherein n is 5.

18. The method according to claim 8, wherein R₆ is a sulphamate group.

19. The method according to claim 1, wherein the compound is selected from compounds of the Formulae, 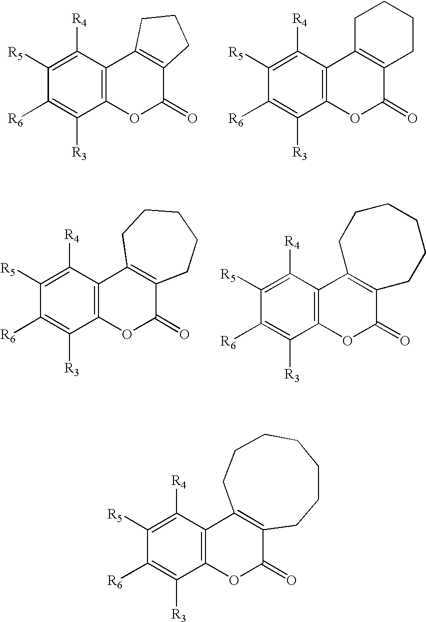
wherein R₃-R₆ are independently selected from H, halo, hydroxy, sulphamate, alkyl and substituted variants or salts thereof; but wherein at least one of R₃-R₆ is a sulphamate group.

20. The method according to claim 7, wherein the sulphamate group has the formula: 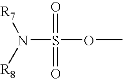
wherein R₇ and R₈ are independently selected from H, alkyl, cycloalkyl, alkenyl, acyl and aryl, or combinations thereof, or together represent alkylene, wherein the or each alkyl or cycloalkyl or alkenyl or optionally contain one or more hetero atoms or groups.

21. The method according to claim 20 wherein at least one of R₇ and R₈ is H.

22. The method according to claim 20 wherein each of R₇ and R₈ is H.

23. The method according to claim 1, wherein the compound is selected from compounds of the Formulae

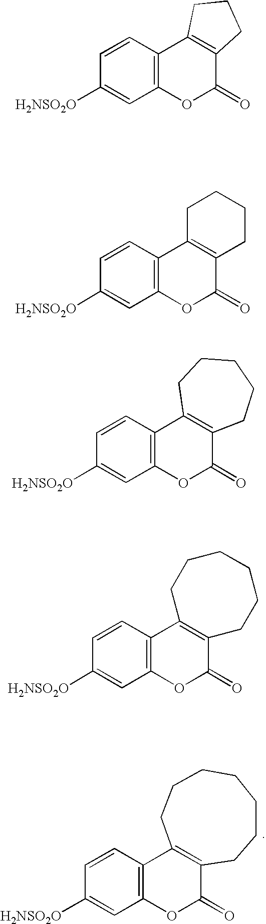

24. The method according to claim 1, wherein the compound is [chemical structure]
